(19) United States
(12) Patent Application Publication
(10) Pub. No.: US 2026/0365625 A1
(43) Pub. Date: Nov. 19, 2026

(54) DEUTERIUM ENRICHED PHENYL DERIVATIVES FOR TREATING MITOCHONDRIARELATED DISORDERS OR CONDITIONS

(71) Applicant: Rivus Pharmaceuticals, Inc., Charlottesville, VA (US)

(72) Inventor: Shaharyar Khan, Charlottesville, VA (US)

(21) Appl. No.: 19/131,038

(22) PCT Filed: Nov. 20, 2023
(86) PCT No.: PCT/US2023/080524

§ 371 Date: May 19, 2025

Related U.S. Application Data
(60) Provisional application No. 63/384,542, filed on Nov. 21, 2022.

(51) Int. Cl. C07D 233/91; A61K 31/4168; A61P 35/00; C07B 59/00

(52) U.S. Cl. CPC C07D 233/91; A61K 31/4168; A61P 35/00; C07B 59/002

(57) ABSTRACT

A deuterated compound is disclosed. A method of controlling various diseases by administration of the deuterated compound is disclosed.

### RELATED APPLICATIONS

This patent application claims the benefit of U.S. Provisional Patent Application No. 63/384,542, filed Nov. 21, 2022, which is incorporated by reference herein and relied upon for all purposes.

### TECHNICAL FIELD

The present disclosure provides novel, deuterium-enriched controlled metabolic accelerator compounds or pharmaceutically acceptable salt thereof, for treating mitochondria-related disorders or conditions.

### BACKGROUND

Cardiovascular diseases are type of diseases that involve the heart and blood vessels, such as coronary artery diseases, hear attack, stroke, heart failure, hypertensive heart disease, and rheumatic heart disease. More than 6.5 million people in the United States have heart failure (Cardiology Today, Apr. 6, 2017). Heart failure with preserved ejection fraction (HFpEF), also known as diastolic heart failure, causes almost one-half of the 6.5 million cases of heart failure in the United States. HFpEF results from abnormalities of active ventricular relaxation and passive ventricular compliance, leading to a decline in stroke volume and cardiac output (Am Fam Physician. 2017 Nov. 1; 96(9):582-588). In heart failure with reduced ejection fraction (HFrEF), also known as systolic heart failure, the heart muscle is not able to contract adequately and expels less oxygen-rich blood into the body. Mortality was similar between patients with HFpEF and HFrEF (Cardiology Today, Apr. 6, 2017). Heart failure with midrange ejection fraction (HFmrEF) is a new category of heart failure, in between HFpEF and HFrEF. HFmrEF has a prevalence of 10-20% of heart failure patients (Maedica (Bucur), 2016, 11(4): 320-324). Therefore, there is a great need for effective treatments of heart failure including HFpEF, HFrEF, and HFmrEF.

Obesity is a well-known risk factor for the development of many common diseases such as type 2 diabetes (T2D) and non-alcoholic fatty liver disease (NAFLD). Obesity may be counteracted by increasing the energy expenditure and calorie consumption, or decreasing the calorie intake. There is a need for pharmaceutical agents that are capable of controlling excess adipose tissue, for instance, by increasing the energy expenditure or decreasing the energy intake.

Mitochondria control metabolism in individual cells by burning sugars and fats. Mitochondrial uncoupling is a robust and natural process that the body utilizes to generate heat. Heat is generated by the mitochondrion via the uncoupling of respiration (Complexes I-IV) from ATP phosphorylation (Complex V). In fact, 20-40% of the calories consumed go toward the generation of body heat. Mitochondria-related disorders or conditions occur when mitochondria fail to produce enough energy for the body to function properly, affecting almost any part of the body including the cells of the brain, adipose tissue, nerves, muscles, heart, lungs, liver, kidneys, pancreas, eyes, and ears.

5-[(2,4-Dinitrophenoxy)methyl]-1-methyl-2-nitro-1H-imidazole is a novel small molecule uncoupler. It works as a controlled metabolic accelerator (CMA). It is designed to effectively address the root cause of metabolic diseases, the accumulation of fat and sugars in the body. CMAs work to improve cellular metabolism and increase energy expenditure and calorie consumption, reducing the accumulation of fat. Using a new controlled and targeted approach, 5-[(2,4-dinitrophenoxy)methyl]-1-methyl-2-nitro-1H-imidazole can increase mitochondrial proton leak, an ongoing process in the body that dissipates energy, and accounts for 20%-40% of daily calories. 5-[(2,4-Dinitrophenoxy)methyl]-1-methyl-2-nitro-1H-imidazole leverages a mitochondrial uncoupling mechanism to increase substrate utilization.

Accordingly, there is a need for uncouplers that can safely treat mitochondria-related disorders or conditions. The present disclosure provides a method of treating a subject afflicted with mitochondria-related disorders or conditions, such as metabolic disorders including obesity, diabetes, or diabetes-associated complications. The novel deuterium-enriched controlled metabolic accelerator compounds disclosed herein satisfy these needs in whole or in part.

### SUMMARY

The present disclosure includes a novel deuterium-enriched compound of formula (I):

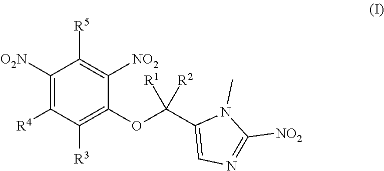

or a pharmaceutically acceptable salt thereof, wherein R¹-R⁵ are independently H or D, provided that at least one of R¹-R⁵ is D.

The compound of formula (I) may be a controlled metabolic accelerator (CMA). CMAs generally improve cellular metabolism and increase energy expenditure and calorie consumption.

In some embodiments, the present disclosure provides a method for treating a cardiovascular disease in a subject comprising administering to the subject a therapeutically effective amount of the compound of formula (I) or a pharmaceutically acceptable salt thereof.

In some embodiments, the present disclosure provides a method of reducing a cardiovascular risk or mortality in a subject suffering from a symptom due to a cardiovascular disease, comprising administering to the subject a therapeutically effective amount of the compound of formula (I) or a pharmaceutically acceptable salt thereof.

In some embodiments, the cardiovascular disease is selected from the group consisting of heart failure, heart attack, coronary artery disease, and coronary heart disease (CHD).

In some embodiments, the cardiovascular disease is selected from the group consisting of heart failure, heart attack, coronary artery disease, and coronary heart disease (CHD).

Heart failure as used herein includes heart failure with preserved ejection fraction (HFpEF), or heart failure with reduced ejection fraction (HFrEF), or heart failure with mid-range ejection fraction (HFmrEF).

In some embodiments, the cardiovascular disease is HFpEF.

In some embodiments, the cardiovascular disease is HFrEF.

In some embodiments, the cardiovascular disease is HFmrEF.

In some embodiments, the present disclosure provides a method for treating heart failure with preserved ejection fraction (HFpEF) in a subject, comprising administering to the subject a therapeutically effective amount of the compound of formula (I) or a pharmaceutically acceptable salt thereof.

In some embodiments, the present disclosure provides a method for treating heart failure with reduced ejection fraction (HFrEF) in a subject, comprising administering to the subject a therapeutically effective amount of the compound of formula (I), or a pharmaceutically acceptable salt thereof.

In some embodiments, the present disclosure provides a method for treating heart failure with mid-range ejection fraction (HFmrEF) in a subject, comprising administering to the subject a therapeutically effective amount of the compound of formula (I) or a pharmaceutically acceptable salt thereof.

In some embodiments, the subject is suffering from at least one of shortness of breath, shortness of breath with exertion, impaired energetics in the heart, dizziness, fatigue, dyspnea, palpitations (atrial fibrillation), chest discomfort, edema, syncope, and a limit on an activity of daily living. In some embodiments, the limit on an activity of daily living is difficulties on personal care, mobility, and eating.

In some embodiments, the subject is suffering from symptoms selected from reduced exercise tolerance, fatigue, tiredness, increased time to recover after exercise, and ankle swelling.

In some embodiments, the subject is suffering from at least one of coronary artery disease, hypertension, and heart murmur.

In some embodiments, the present disclosure provides a method of reducing blood pressure in a subject comprising administering to the subject a therapeutically effective amount of the compound of formula (I), or a pharmaceutically acceptable salt thereof.

In some embodiments, the subject is suffering from at least one of cardiovascular disease, hypertension, resistant hypertension, and severe hypertension.

In some embodiments, the cardiovascular disease is heart failure (which may be HFpEF, HFrEF, or HFmrEF), heart attack, coronary artery disease, or coronary heart disease (CHD). In some embodiments, the subject has hypertension associated with HFpEF.

In some embodiments, the subject has hypertension associated with HFrEF.

In some embodiments, the subject has hypertension associated with HFmrEF.

In some embodiments, wherein the subject experiences a reduction of blood pressure of at least 5 mmHg after the administration of the compound of formula (I) or a pharmaceutically acceptable salt thereof.

In some embodiments, the method reduces the risk of developing a cardiovascular disease, and/or reduces the risk of HFpEF, HFrEF, or HFmrEF.

In some embodiments, the method slows the progression of HFpEF, HFrEF, or HFmrEF. In some embodiments, the method comprises at least one of: i) extending the half-life (t½) of DNP; ii) delaying the time to maximum plasma concentration (Tₘₐₓ) of DNP; iii) lowering maximum plasma concentration (Cₘₐₓ) of DNP; and iv) increasing area under the curve (AUC).

In some embodiments, the subject does not experience significant systemic toxicity, side effects, significant increase in body temperature, or significant increase in heart rate after administration.

In some embodiments, the present disclosure provides a method of treating a cardiovascular disease by achieving: i) asteady state of maximum plasma concentration (Cmax) of DNP from about 80 ng/mL to about 8300 ng/mL; ii) mean half-life (t½) of DNP about 20-50 hours, about 25-40 hours, or about 30-40 hours; iii) median time to maximum plasma concentration (Tₘₐₓ) of DNP about 6-8 hours or about 6-10 hours; iv) median area under the curve extrapolated to infinity (AUC_{mf}) of DNP about 3 h*μg/mL to about 420 h*μg/mL; and AUC/Cmax ratio of about 18, by administering to a subject about 30 mg to about 1400 mg of a compound of formula (I) or a pharmaceutically acceptable salt thereof.

In some embodiments, the present disclosure provides a method of treating mitochondria-related disorders or conditions without causing a clinically significant risk of adverse events in a subject, the method comprising administering to the subject a therapeutically effective amount of the compound of formula (I) or a pharmaceutically acceptable salt thereof.

In some embodiments, the present disclosure provides a method of reducing toxicity or side effects in treating mitochondria-related disorders or conditions in a subject, comprising administering to the subject a therapeutically effective amount of a compound of formula (I) or a pharmaceutically acceptable salt thereof.

In some embodiments, the present disclosure provides a method of preventing overdose in treating mitochondria-related disorders or conditions in a subject, comprising administering to the subject a therapeutically effective amount of a compound of formula (I) or a pharmaceutically acceptable salt thereof.

In some embodiments, the present disclosure provides a method for increasing metabolic rate or resting energy expenditure without causing a clinically significant risk of adverse events in a subject, comprising administering to the subject a therapeutically effective amount of a compound of formula (I) or a pharmaceutically acceptable salt thereof.

In some embodiments, the present disclosure provides a method for treating dysmetabolism in a subject comprising administering to the subject a therapeutically effective amount of the compound of formula I, or a pharmaceutically acceptable salt thereof. In some embodiments, the present disclosure provides a method of treating hypertriglyceridemia associated with cardiovascular disease, atherosclerosis, obesity, hypertension, diabetes, insulin resistance, and/or liver disease in a subject, comprising administering to the subject a therapeutically effective amount of the compound of formula (I), or a pharmaceutically acceptable salt thereof.

In some embodiments, the present disclosure provides a method of treating severe hypertriglyceridemia in a subject, comprising administering to the subject a therapeutically effective amount of the compound of formula (I), or a pharmaceutically acceptable salt thereof.

In some embodiments, the present disclosure provides a method of reducing liver fat by at least 50%; or a method of reducing lipids by at least 10% in a subject; the method comprising administering to the subject a therapeutically effective amount of the compound of formula (I), or a pharmaceutically acceptable salt thereof.

In some embodiments, the present disclosure provides a method of treating obesity, excess body fat, type 2 diabetes, insulin resistance or intolerance, high blood pressure, dyslipidemia, atherosclerosis, hypertriglyceridemia, acquired lipodystrophy, inherited lipodystrophy, partial lipodystrophy, metabolic syndrome, Rett's syndrome, metabolic syndrome associated with aging, metabolic diseases associated with increased reactive oxygen species (ROS), Friedreich's ataxia, NAFLD, NASH, noncirrhotic NASH, noncirrhotic NASH with liver fibrosis, hepatic steatosis, hepatic fibrosis, liver cirrhosis, or hepatocellular carcinoma, the method comprising administering a therapeutically effective amount of the compound of formula (I) or a pharmaceutically acceptable salt thereof in a subject, to achieve at least one of: i) a steady state of maximum plasma concentration (Cₘₐₓ) of a phenolic metabolite (“metabolite”) of the compound of formula (I) from about 80 ng/ml to about 8300 ng/ml; ii) mean half-life (t½) of the metabolite of about 20-50 hours, about 25-40 hours, or about 30-40 hours; iii) median time to maximum plasma concentration (Tₘₐₓ) of the metabolite of about 6-8 hours or about 6-10 hours; iv) median area under the curve extrapolated to infinity (AUCinf) of the metabolite of about 3 h*μg/mL to about 420 h*μg/mL; and v) AUC/Cₘₐₓ ratio of about 18.n some embodiments, the compound of formula (I) is useful for regulating mitochondria activities, reducing adiposity, treating diseases including metabolic disorders, diabetes or diabetes-associated complications such as heart disease and renal failure, and moderating or controlling of weight gain in a subject.

In some embodiments, the disorder is metabolic disorders, diabetes, or diabetes-associated complications, such as heart disease and renal failure, and moderating or controlling of weight gain in a subject.

In some embodiments, the disorder is obesity or excess body fat.

In some embodiments, the disorder is diabetes.

In some embodiments, the disorder is type 2 diabetes (T2DM).

In some embodiments, the disorder is non-alcoholic fatty liver disease (NAFLD).

In some embodiments, the disorder is non-alcoholic steatohepatitis (NASH).

In some embodiments, the disorder is hepatic steatosis.

In some embodiments, the disorder is insulin resistance or intolerance.

In some embodiments, the disorder is dyslipidemia.

In some embodiments, the disorder is cardiovascular disease.

In some embodiments, the disorder is atherosclerosis.

In some embodiments, the compound of formula (I) or a pharmaceutically acceptable salt thereof is used to reduce adiposity, controlling or preventing of weight gain in a subject, and/or to stimulate oxygen consumption rate (OCR) in a subject, and/or to treat inflammation and fibrosis resulting in NASH in a subject.

### BRIEF DESCRIPTION OF THE DRAWINGS

FIG. 1 shows a nuclear magnetic resonance spectrum of a compound according to the disclosure, also known as Compound (A).

FIG. 2 illustrates Compound (A)'s effect on oxygen consumption rate.

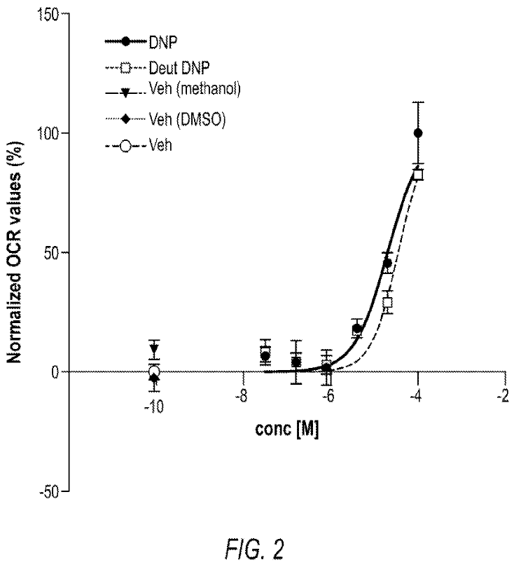

### DETAILED DESCRIPTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. The following definitions are provided to facilitate understanding of certain terms used frequently herein and are not meant to limit the scope of the present disclosure.

As used herein, an effective amount is defined as the amount required to confer a therapeutic effect on the treated subject, and is typically determined based on age, surface area, weight, and condition of the subject.

The terms “a” or “an,” as used in herein means one or more.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

As used herein, the term “about” means a range of values including the specified value, which a person of ordinary skill in the art would consider reasonably similar to the specified value. In some embodiments, the term “about” means within a standard deviation using measurements generally acceptable in the art. In some embodiments, “about” means a range extending to +/−10%, +/−5%, or +/−2% of the specified value. In some embodiments, “about” means the specified value.

As used herein, “treatment” or “treating” or “palliating” or “ameliorating” or “reducing” or “controlling” are used interchangeably herein. These terms refer to an approach for obtaining beneficial or desired results including but not limited to a therapeutic benefit. By therapeutic benefit means eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. Treatment includes causing the clinical symptoms of the disease to slow in development by administration of a composition; suppressing the disease, that is, causing a reduction in the clinical symptoms of the disease; inhibiting the disease, that is, arresting the development of clinical symptoms by administration of a composition after the initial appearance of symptoms; and/or relieving the disease, that is, causing the regression of clinical symptoms by administration of a composition after their initial appearance.

“Patient” or “subject” or “subject in need thereof” refers to a living organism suffering from or prone to a disease or condition that can be treated by using the methods provided herein. The term does not necessarily indicate that the subject has been diagnosed with a particular disease, but typically refers to an individual under medical supervision. Non-limiting examples include humans, other mammals, bovines, rats, mice, dogs, cats, monkeys, goat, sheep, cows, deer, and other non-mammalian animals. In some embodiments, a patient, subject or subject in need thereof is a human.

As used herein, “administration” of a disclosed compound encompasses the delivery to a subject of a compound as described herein, or a prodrug or other pharmaceutically acceptable derivative thereof, using any suitable formulation or route of administration, e.g., as described herein.

“Pharmaceutically acceptable” refers to compounds, salts, compositions, dosage forms and other materials that are useful in preparing a pharmaceutical composition that is suitable for veterinary or human pharmaceutical use.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof. Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, b-hydroxybutyric, salicylic, galactaric and galacturonic acid. Suitable pharmaceutically acceptable base addition salts of compounds of the invention include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

An “effective amount” is an amount sufficient to accomplish a stated purpose (e.g. achieve the effect for which it is administered, treat a disease, reduce enzyme activity, reduce one or more symptoms of a disease or condition, reduce viral replication in a cell). An example of an “effective amount” is an amount sufficient to contribute to the treatment, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). Efficacy can also be expressed as “-fold” increase or decrease. For example, a therapeutically effective amount can have at least a 1.2-fold, 1.5-fold, 2-fold, 5-fold, or more effect over a control.

As used herein, the term “significant increase in body temperature” in a subject refers to a body temperature increase that is associated with deleterious effects on the subject, not limited to illness, physical discomfort or pain, coma and death. In one non-limiting embodiment, the significant increase in body temperature is an increase of about 0.5 °C, about 1 °C, about 1.5 °C, about 2 °C, about 2.5 °C, about 3 °C, about 3.5 °C, about 4 °C, about 4.5 °C, about 5 °C, about 5.5 °C, about 6 °C or higher. In another non-limiting embodiment, the significant increase in body temperature lasts for about 5 min, about 15 min, about 30 min, about 45 min, about 1 h, about 1.5 h, about 2 h, about 3 h, about 4 h, about 5 h, about 6 h, about 7 h, about 8 h, about 9 h, about 10 h, about 12 h, about 14 h, about 16 h, about 18 h, about 20 h, about 22 h, about 24 h or longer.

As used herein, the term “significant systemic toxicity” in a subject refers to systemic toxicity that is associated with deleterious effects on the subject, not limited to illness, physical discomfort or pain, coma and death. In one non-limiting embodiment, significant systemic toxicity is indicated by increase in levels of liver enzymes, blood urea nitrogen or creatinine as compared to the corresponding levels in the subject in the absence of administration of the composition.

In some embodiments, the present disclosure provides a method for treating a cardiovascular disease in a subject comprising administering to the subject a therapeutically effective amount of the compound of formula (I) or a pharmaceutically acceptable salt thereof. Initial studies suggest that effective amount of the compound of formula (I) or a pharmaceutically acceptable salt thereof can achieve the following:
Controlled and improved metabolite control—low Cmax, high AUC, low variability.
Potential once daily oral administration.
Liver specific uncoupling and reduced systemic effects.
A wide therapeutic index.

The compound of formula (I) may be useful in treating a wide range of diseases safely.

In some embodiments, the present disclosure provides a method of treating heart failure in a subject suffering from a symptom due to a cardiovascular disease, comprising administering to the subject a therapeutically effective amount of the compound of formula (I) or a pharmaceutically acceptable salt thereof.

In some embodiments, the present disclosure provides a method of reducing a cardiovascular risk or mortality in a subject suffering from a symptom due to a cardiovascular disease, comprising administering to the subject a therapeutically effective amount of the compound of formula (I) or a pharmaceutically acceptable salt thereof.

In some embodiments, the symptom due to a cardiovascular disease is shortness of breath, dizziness, chest pain, syncope, fatigue, or limits on activities of daily living.

In some embodiments, the limit on an activity of daily living is difficulties on personal care, mobility, or eating.

In some embodiments, the present disclosure provides a method of treating cardiovascular diseases in a subject, comprising administering to the subject a therapeutically effective amount of the compound of formula (I) or a pharmaceutically acceptable salt thereof.

In some embodiments, the cardiovascular diseases are associated with obesity. In some embodiments, the cardiovascular diseases include the following diseases, disorders, or conditions.

Disrupted cardiovascular hemodynamics characterized by increased heart rate among those with physical inactivity, increased risk for atrial fibrillation, increased blood volume, increased cardiac output, increased systemic vascular resistance among those with hypertension and insulin resistance, increased arterial pressure, increased left ventricular wall stress, increased pulmonary artery pressure, alternations in ventricular pressure among those with sleep apnea. Atherosclerosis and myocardial infarction which may increase indirectly through promotion of major atherosclerotic risk factors (e.g., diabetes mellitus, hypertension, dyslipidemia) or directly through adiposopathic endocrinopathies and immunopathies of epicardial adipose tissue.

Epicardial fat accumulation, pathogenic paracrine and vasocrine signaling, increased inflammatory macrophages, increased T-Lymphocytes and mast cells, increased adiposopathic adipokines, and reduced vasculoprotective adipokines

Heart failure (HF), especially HF with preserved ejection fraction (HFpEF)

Atherosclerotic Cardiovascular Disease (ASCVD), dysrhythmias, fatty infiltration of the heart, increased coronary calcium.

Sleep apnea that may lead to hypoxia, increased epinephrine (adrenaline) may lead to high blood pressure, swings in thoracic pressures increase left and right heart ventricular pressure.

Thrombosis and thromboembolic events, increased adipose tissue compresses pelvic and lower extremities veins, impairs venous return, and promotes deep vein thrombosis.

Abnormal heart cell and structure characterized by myocardial steatosis, apoptosis and fibrosis as well as left ventricular remodeling and hypertrophy, left atrial enlargement, right ventricular hypertrophy and increased pericardial and perivascular adipose tissue.

Reduced heart function characterized by hypoxia due to sleep apnea, atherosclerosis, thrombosis, left ventricular dysfunction (both diastolic and systolic) and right ventricular failure. [0101] Immunopathies characterized by increased pro-inflammatory adipocytokines e.g. tumor necrosis factor (TNF), interleukins such as interleukin-6 (IL-6), monocyte chemoattractant protein-1 (MCP-1) or C-reactive protein (CRP) or decreased anti-inflammatory adipocytokines (e.g., adiponectin) and IL-10.

Immonopathies characterized by increased neutrophilic activation and granulation such as severe asthma, and glucocorticoid resistant severe asthma.

Endocrinopathies characterized by activation of the renin-angiotensin-aldosterone system leading to elevated blood pressure, alteration of peroxisome proliferator activated receptor expression.

Endocrinopathies characterized by hyperinsulinemia, systemic insulin resistance and adiposopathy, myocardial insulin insensitivity.

Endocrinopathies characterized by leptin insensitivity, with increased leptin levels potentially contributing to cardiac hypertrophy and heart failure.

Lipotoxicity characterized by limitations of energy storage in peripheral subcutaneous adipose tissue.

Overflow of free fatty acid delivery to liver, muscle, pancreas, kidney and/or visceral, pericardial, and perivascular adipose tissue

In some embodiments, the cardiovascular disease is heart failure, heart attack, coronary artery disease, and coronary heart disease (CHD).

In some embodiments, heart failure includes HFpEF, HFrEF, or HFmrEF.

In some embodiments, the subject experiences a reduction in the risk of a major cardiovascular event after administration.

In some embodiments, the major cardiovascular event is death or hospitalization for worsening of the disease.

In some embodiments, the present disclosure provides a method for treating heart failure with preserved ejection fraction (HFpEF) in a subject, comprising administering to the subject a therapeutically effective amount of the compound of formula (I) or a pharmaceutically acceptable salt thereof. In some embodiments, the present disclosure provides a method for treating heart failure with reduced ejection fraction (HFrEF) in a subject, comprising administering to the subject a therapeutically effective amount of the compound of formula (I) or a pharmaceutically acceptable salt thereof. In some embodiments, the present disclosure provides a method for treating heart failure with mid-range ejection fraction (HFmrEF) in a subject, comprising administering to the subject a therapeutically effective amount of the compound of formula (I) or a pharmaceutically acceptable salt thereof. In some embodiments, the subject suffers from obesity, excess body fat, diabetes, high blood pressure (hypertension), dyslipidemia, hypertriglyceridemia, acquired lipodystrophy, inherited lipodystrophy, partial lipodystrophy, or metabolic syndrome.

In some embodiments, the subject is suffering from at least one symptoms selected from shortness of breath, shortness of breath with exertion, impaired energetics in the heart, dizziness, fatigue, dyspnea, palpitations (atrial fibrillation), chest discomfort, edema, syncope, and a limit on an activity of daily living.

In some embodiments, the limit on an activity of daily living is difficulties on personal care, mobility, and eating.

In some embodiments, the subject is suffering from at least one of reduced exercise tolerance, fatigue, tiredness, increased time to recover after exercise, and ankle swelling.

In some embodiments, the subject is suffering from at least one of coronary artery disease, hypertension, and heart murmur.

In some embodiments, wherein the subject experiences an improvement of cardiac bioenergetic deficiency after administration, wherein the improvement comprises weight loss >5%, reduction in blood pressure, increased quality of life, increased exercise tolerance, and/or a reduction in the risk of a major cardiovascular event, wherein the major cardiovascular event is selected from the group consisting of death, hospitalization for worsening of the disease, and myocardial infraction.

In some embodiments, the method further comprises assessing peak oxygen consumption (VO2) and/or VE/CO2 or VE/VCO2 slope in the subject during exercise before and after administration of the therapeutically effective amount of the compound of formula (I), wherein an increase in VO2 in the subject after administration indicates a reduction in the extent of HFpEF, HFrEF, HFmrEF, or one or more symptomatic component or condition of cardiovascular diseases thereof in the subject.

In some embodiments, the method increases VO2 in the subject after administration. In some embodiments, the method increases the subjects exercise tolerance. In some embodiments, the method increases the subjects exercise tolerance as measured by assessing 6-minute walk distance (6MWD) before and after administration of the therapeutically effective amount of the compound of formula (I), wherein an increase in 6MWD in the subject after administration indicates a reduction in the extent of HFpEF or the at least one symptomatic component or condition thereof in the subject.

In some embodiments, the method increases 6MWD after the administration.

In some embodiments, the HFpEF in the subject is diagnosed according to echocardiography (E/e′) or biomarkers (NT-proBNP).

In some embodiments, the method further comprises assessing a NYHA classification score of the subject before and after administration.

The NYHA functional classification grades the severity of heart failure symptoms as one of four functional classes. The NYHA functional classification is widely used in clinical practice and in research because it provides a standard description of severity that can be used to assess response to treatment and to guide management. The NYHA functional classification based on severity of symptoms and physical activity are:
Class I: No limitation of physical activity. Ordinary physical activity does not cause undue breathlessness, fatigue, or palpitations
Class II: Slight limitation of physical activity. Comfortable at rest, but ordinary physical activity results in undue breathlessness, fatigue, or palpitations.
Class III: Marked limitation of physical activity. Comfortable at rest, but less than ordinary physical activity results in undue breathlessness, fatigue, or palpitations.
Class IV: Unable to carry on any physical activity without discomfort. Symptoms at rest can be present. If any physical activity is undertaken, discomfort is increased.

In some embodiments, the method further comprises the step of: assessing a NYHA classification score of the subject before and after administration of the therapeutically effective amount of the compound of formula (I), or a pharmaceutically acceptable salt thereof, wherein a decreased NYHA score after administration indicates a reduction in the extent of the disease in the subject.

In some embodiments, the method decreases the NYHA classification score of the subject after administration from Class III to Class II, or from Class II to Class I.

In some embodiments, the method increases the subject's quality of life. In some embodiments, the method increases the subjects quality of life as assessed by a standardized questionnaire such as KCCQ (Kansas City Cardiomyopathy Questionnaire), KCCQ-12, KCCQ-Physical Limitation Score (KCCQ-PLS), KCCQ-Totally Symptom Score (KCCQ-TSS) KCCQ-Clinical Summary Score (KCCQ-CSS), KCCQ-Overall Summary Score (KCCQ-OSS) or other derivatives.

In some embodiments, the present disclosure provides a method of reducing blood pressure in a subject comprising administering to the subject a therapeutically effective amount of the compound of formula (I), or a pharmaceutically acceptable salt thereof.

In some embodiments, the subject is suffering from at least one of: cardiovascular disease, hypertension, resistant hypertension, and severe hypertension.

In some embodiments, the cardiovascular disease is selected from the group consisting of heart failure, HFpEF, HFrEF, heart attack, coronary artery disease, and coronary heart disease (CHD).

In some embodiments, the subject has hypertension associated with HFpEF.

In some embodiments, the subject has hypertension associated with HFrEF.

In some embodiments, the subject has hypertension associated with HFmrEF.

In some embodiments, the subject is suffering from at least one of symptoms selected from headaches, shortness of breath, chest pain, nosebleeds, dizziness, fatigue, vision problem, irregular heartbeat, blood in urine, sweating, trouble sleeping, and blood spots in eyes.

In some embodiments, the symptoms are associated with HFpEF, HFrEF, or HFmrEF. In some embodiments, wherein the reducing blood pressure comprises reducing diastolic blood pressure and/or reducing systolic blood pressure.

In some embodiments, wherein the subject experiences a reduction of blood pressure of at least 5 mmHg after the administration.

In some embodiments, the method reduces the risk of developing a cardiovascular disease, reduces the risk of HFpEF, or slows the progression of HFpEF.

In some embodiments, the method reduces the risk of developing a cardiovascular disease, reduces the risk of HFrEF, or slows the progression of HFrEF.

In some embodiments, the method reduces the risk of developing a cardiovascular disease, reduces the risk of HFmrEF, or slows the progression of HFmrEF.

In some embodiments, the subject is in a fasted condition before administration. In some embodiments, the subject is in a fed condition before administration.

In some embodiments, the subject experiences a reduction in at least one of body weight, blood pressure, and blood glucose after the administration.

In some embodiments, the subject experiences at least one of: i) a reduction of body weight by at least 5% or at least 10%; ii) a reduction of blood pressure of at least 5 mmHg; iii) a reduction of HbAic by at least 0.5%, or at least 1.5%; iv) a reduction of lipids by at least 10%; and v) a reduction of liver fat by at least 50%.

In some embodiments, the method comprises at least one of: extending the half-life (ti/2) of the metabolite; delaying the time to maximum plasma concentration (Tmax) of a phenolic metabolite; lowering maximum plasma concentration (Cmax) of the metabolite; and increasing area under the curve (AUC).

In some embodiments, the mean half-life of a phenolic metabolite is extended to about 20-50 hours, 25-40 hours, or 30-40 hours.

In some embodiments, the median Tmax of a phenolic metabolite is extended to at least 6 hours or at least 8 hours.

In some embodiments, the median Tmax of a phenolic metabolite is extended to about 6-8 hours or about 6-10 hours.

In some embodiments, lowering a phenolic metabolite Cmax comprises providing a steady state of Cmax of a phenolic metabolite from about 80 ng/mL to about 8300 ng/ml in the subject after administration.

In some embodiments, the method provides an AUC/Cmax ratio of about 18 in the subject. In some embodiments, the subject does not experience significant systemic toxicity, side effects, significant increase in body temperature, or significant increase in heart rate after administration.

In some embodiments, the side effects comprise at least one of nausea, vomiting, sweating, dizziness, headaches, cataracts, glaucoma, pyrexia, hyperthermia, tachycardia, diaphoresis, tachypnoea, and death. In some embodiments, the present disclosure provides a method of treating a cardiovascular disease, the method comprising administering to a subject about 30 mg to about 1400 mg of the compound of formula (I) or a pharmaceutically acceptable salt thereof to achieve at least one of: i) a steady state of maximum plasma concentration (Cmax) of a phenolic metabolite from about 80 ng/ml to about 8300 ng/mL; ii) mean half-life (t½) of a phenolic metabolite about 20-50 hours, about 25-40 hours, or about 30-40 hours; iii) median time to maximum plasma concentration (Tmax) of a phenolic metabolite about 6-8 hours or about 6-10 hours; iv) median area under the curve extrapolated to infinity (AUCmf) of a phenolic metabolite about 3 h*μg/mL to about 420 h*μg/mL; and v) AUC/Cₘₐₓ ratio of about 18.

In some embodiments, the present disclosure provides a method of treating mitochondria-related disorders or conditions without causing a clinically significant risk of adverse events in a subject, the method comprising administering to the subject a therapeutically effective amount of the compound of formula (I) or a pharmaceutically acceptable salt thereof.

In some embodiments, the present disclosure provides a method of reducing toxicity or side effects in treating mitochondria-related disorders or conditions in a subject, comprising administering to the subject a therapeutically effective amount of the compound of formula (I) or a pharmaceutically acceptable salt thereof.

In some embodiments, the present disclosure provides a method of reducing toxicity or side effects of a phenolic metabolite in treating mitochondria-related disorders or conditions in a subject, comprising administering to the subject a therapeutically effective amount of the compound of formula (I) or a pharmaceutically acceptable salt thereof.

In some embodiments, the present disclosure provides a method of preventing overdose in treating mitochondria-related disorders or conditions in a subject, comprising administering to the subject a therapeutically effective amount of the compound of formula (I) or a pharmaceutically acceptable salt thereof.

In some embodiments, the present disclosure provides a method of preventing overdose of a phenolic metabolite in treating mitochondria-related disorders or conditions in a subject, comprising administering to the subject a therapeutically effective amount of the compound of formula (I) or a pharmaceutically acceptable salt thereof.

In some embodiments, the mitochondria-related disorder comprises obesity, excess body fat, diabetes, insulin resistance or intolerance, high blood pressure, dyslipidemia, cardiovascular disease, atherosclerosis, hypertriglyceridemia, acquired lipodystrophy, inherited lipodystrophy, partial lipodystrophy, metabolic syndrome, Rett's syndrome, metabolic syndrome associated with aging, metabolic diseases associated with increased reactive oxygen species (ROS), Friedreich's ataxia, or liver disease.

In some embodiments, the disorder are Branched Chain Amino Acid (BCAA) metabolism disorders, lysosomal storage disorders, glycogen storage disorders.

In some embodiments, the diabetes is type 2 diabetes (T2DM).

In some embodiments, the cardiovascular disease comprises heart failure, HFpEF, HFrEF, HFmrEF, heart attack, coronary artery disease, or CHD.

In some embodiments, the liver disease comprises non-alcoholic fatty liver disease (NAFLD), non-alcoholic steatohepatitis (NASH), noncirrhotic NASH, noncirrhotic NASH with liver fibrosis, hepatic steatosis, hepatic fibrosis, liver cirrhosis, or hepatocellular carcinoma. [0169] In some embodiments, the mitochondria-related disorder comprises cardiovascular disease, hypertension, type 2 diabetes, dyslipidemia, obesity, or non-alcoholic steatohepatitis (NASH).

In some embodiments, the mitochondria-related condition is at least on of steatosis, inflammation, fibrosis, cirrhosis, and hepatocyte injury in NASH.

In some embodiments, the toxicity, adverse events, side effects, and overdose are associated with a mitochondria uncoupler.

In some embodiments, the mitochondria uncoupler is a phenolic metabolite.

In some embodiments, the method comprises at least one of: i) extending the half-life (t½) of a phenolic metabolite; ii) delaying the time to maximum plasma concentration (Tmax) of a phenolic metabolite; iii) lowering maximum plasma concentration (Cmax) of a phenolic metabolite; and iv) increasing the area under the curve (AUC).

In some embodiments, the present disclosure provides a method for increasing metabolic rate without causing a clinically significant risk of adverse events in a subject, comprising administering to the subject a therapeutically effective amount of the compound of formula (I) or a pharmaceutically acceptable salt thereof.

In some embodiments, the present disclosure provides a method for increase resting energy expenditure in a subject comprising administering to the subject a therapeutically effective amount of the compound of formula (I) or a pharmaceutically acceptable salt thereof.

In some embodiments, the present disclosure provides a method for treating dysmetabolism in a subject comprising administering to the subject a therapeutically effective amount of the compound of formula (I) or a pharmaceutically acceptable salt thereof.

In some embodiments, the subject suffers from at least one of obesity, excess body fat, type 2 diabetes, insulin resistance or intolerance, high blood pressure, dyslipidemia, atherosclerosis, hypertriglyceridemia, acquired lipodystrophy, inherited lipodystrophy, partial lipodystrophy, metabolic syndrome, Rett's syndrome, metabolic syndrome associated with aging, metabolic diseases associated with increased reactive oxygen species (ROS), Friedreich's ataxia, NAFLD, NASH, noncirrhotic NASH, noncirrhotic NASH with liver fibrosis, hepatic steatosis, hepatic fibrosis, liver cirrhosis, and hepatocellular carcinoma.

In some embodiments, the method comprises increasing resting metabolic rate without causing a clinically significant risk of adverse events.

In some embodiments, the resting metabolic rate is increased by at least 10%.

In some embodiments, the resting metabolic rate is increased by at least 20%.

In some embodiments, the subject experiences an increase of resting energy expenditure of at least 10% after the administration.

In some embodiments, the subject experiences an increase of resting energy expenditure of at least 20% after the administration.

In some embodiments, the subject experiences an increase of resting energy expenditure of about 30% after the administration.

In some embodiments, the method slows the progression of at least one of atherosclerosis, NAFLD, NASH, noncirrhotic NASH, noncirrhotic NASH with liver fibrosis, hepatic steatosis, hepatic fibrosis, liver cirrhosis, and hepatocellular carcinoma.

In some embodiments, the method accelerates human body's natural processes to improve cardio-metabolic processes. In some embodiments, the present disclosure provides a method of treating hypertriglyceridemia associated with cardiovascular disease, atherosclerosis, obesity, hypertension, diabetes, insulin resistance, and/or liver disease in a subject, comprising administering to the subject a therapeutically effective amount of the compound of formula (I) or a pharmaceutically acceptable salt thereof.

In some embodiments, the subject has moderate hypertriglyceridemia associated with cardiovascular disease, atherosclerosis, obesity, hypertension, diabetes, insulin resistance, and/or liver disease; or severe hypertriglyceridemia associated with cardiovascular disease, atherosclerosis, obesity, hypertension, diabetes, insulin resistance, and/or liver disease.

In some embodiments, the present disclosure provides a method of treating severe hypertriglyceridemia in a subject, comprising administering to the subject a therapeutically effective amount of the compound of formula (I) or a pharmaceutically acceptable salt thereof.

In some embodiments, the subject has a triglyceride blood level above 500 mg/dL.

In some embodiments, the subject has severe hypertriglyceridemia associated with cardiovascular disease, atherosclerosis, obesity, hypertension, diabetes, insulin resistance, and/or liver disease.

In some embodiments, the subject has treatment resistant hypertriglyceridemia.

In some embodiments, the subject has treatment resistant severe hypertriglyceridemia.

In some embodiments, the subject has treatment resistant severe hypertriglyceridemia associated with cardiovascular disease, atherosclerosis, obesity, hypertension, diabetes, insulin resistance, and/or liver disease.

In some embodiments, the subject is suffering from at least one of abdominal pain, pain in the mid-epigastric, chest, or back regions, gastrointestinal pain, difficulty breathing, loss of appetite, nausea, vomiting, inflammation of the pancreas, memory loss, dementia, xanthelasmas, comeal arcus, and xanthomas.

In some embodiments, the subject is an adult male subject.

In some embodiments, the subject is a Hispanic descendant.

In some embodiments, the method comprises lowering low-density lipoprotein cholesterol levels and/or lowering non-high-density lipoprotein cholesterol levels.

In some embodiments, the method comprises at least one of: i) lowering triglyceride levels by at least 5%, at least 10%, or at least 20%; ii) lowering low-density lipoprotein cholesterol levels by at least 5%, at least 10%, or at least 20%; and iii) lowering non-high-density lipoprotein cholesterol levels by at least 5%, at least 10%, or at least 20%.

In some embodiments, the present disclosure provides a method of reducing liver fat by at least 50% in a subject, comprising administering to the subject a therapeutically effective amount of the compound of formula (I) or a pharmaceutically acceptable salt thereof.

In some embodiments, the present disclosure provides a method of reducing lipids by at least 10% in a subject, comprising administering to the subject a therapeutically effective amount of the compound of formula (I) or a pharmaceutically acceptable salt thereof.

In some embodiments, the present disclosure provides a method of treating or reducing the risk of cancer in a subject, comprising administering to the subject a therapeutically effective amount of the compound of formula (I) or a pharmaceutically acceptable salt thereof.

In some embodiments, the cancer includes biliary tract cancer, bladder cancer, brain cancer (i.e., meningiomas), breast cancer (postmenopausal), cervical cancer, colorectal cancer, endometrial/uterine cancer, esophageal cancer, gallbladder cancer, head and neck cancer, kidney/renal cancer, leukemia, liver cancer, multiple myeloma, non-Hodgkin lymphoma, ovarian cancer, pancreatic cancer, stomach cancer and thyroid cancer, and prostate cancer.

In some embodiments, the cancer is associated with obesity, excess body fat, diabetes, high blood pressure, dyslipidemia, metabolic diseases, liver diseases, and/or cardiovascular diseases.

In some embodiments, the present disclosure provides a method of treating obesity, cancer, excess body fat, type 2 diabetes, insulin resistance or intolerance, high blood pressure, dyslipidemia, atherosclerosis, hypertriglyceridemia, acquired lipodystrophy, inherited lipodystrophy, partial lipodystrophy, metabolic syndrome, Rett's syndrome, metabolic syndrome associated with aging, metabolic diseases associated with increased reactive oxygen species (ROS), Friedreich's ataxia, NAFLD, NASH, noncirrhotic NASH, noncirrhotic NASH with liver fibrosis, hepatic steatosis, hepatic fibrosis, liver cirrhosis, or hepatocellular carcinoma, the method comprising administering a therapeutically effective amount of the compound of formula (I) or a pharmaceutically acceptable salt thereof in a subject, to achieve at least one of: i) a steady state of maximum plasma concentration (Cmax) of a phenolic metabolite from about 80 ng/mL to about 8300 ng/ml; ii) median half-life (t½) of a phenolic metabolite about 20-50 hours, about 25-40 hours, or about 30-40 hours; iii) median time to maximum plasma concentration (Tmax) of a phenolic metabolite about 6-8 hours or about 6-10 hours; iv) median area under the curve extrapolated to infinity (AUCmf) of a phenolic metabolite about 3 h*μg/mL to about 420 h*μg/mL; and v) AUC/Cmax ratio of about 18.

In some embodiments, the method further comprises the step of: determining Fibroscan® Vibration-controlled Transient Elastography (VCTE), Fibroscan® Controlled Attenuation Parameter (CAP) score, Magnetic resonance imaging proton density fat fraction (MRI-PDFF), and Enhanced Liver Fibrosis (ELF) score of the subject before and after administration.

In some embodiments, the subject has CAP score of greater than 300 dB/m before administration.

In some embodiments, the subject has at least 8% liver fat by MRI-PDFF before administering.

In some embodiments, the subject has elevated Body Mass Index (BMI).

In some embodiments, the subject has BMI of about 28.0 kg/m2 to about 45.0 kg/m2.

In some embodiments, the diabetes is type 2 diabetes (T2DM).

In some embodiments, the cardiovascular disease comprises heart failure, HFpEF, HFrEF, HFmrEF, heart attack, coronary artery disease, or CHD.

In some embodiments, the liver disease comprises non-alcoholic fatty liver disease (NAFLD), non-alcoholic steatohepatitis (NASH), noncirrhotic NASH, noncirrhotic NASH with liver fibrosis, hepatic steatosis, hepatic fibrosis, liver cirrhosis, or hepatocellular carcinoma. [0213] In some embodiments, the mitochondria-related condition is at least on of steatosis, inflammation, fibrosis, cirrhosis, and hepatocyte injury in NASH.

In some embodiments, the subject is suffering from non-alcoholic fatty liver disease (NAFLD), non-alcoholic steatohepatitis (NASH), and/or hepatic steatosis.

In some embodiments, the subject suffers from type 2 diabetes, obesity, HFpEF, HFrEF, NAFLD, and/or NASH. In some embodiments, the subject suffers from inflammation, fibrosis, cirrhosis in liver. In some embodiments, the subject does not experience significant systemic toxicity, serious side effects, a clinically significant risk of adverse events, and/or overdoes after administration.

In some embodiments, the toxicity, adverse events, and side effects are associated with a mitochondria uncoupler.

In some embodiments, the mitochondria uncoupler is a phenolic metabolite.

In some embodiments, the subject does not experience a clinically significant risk of adverse events, side effects, toxicity, and/or overdoes associated with a phenolic metabolite. Thus, the subject in need can be safely treated without the danger of serious side effects and overdose.

In some embodiments, the adverse events or side effects comprise at least one of nausea, vomiting, sweating, dizziness, headaches, cataracts, glaucoma, pyrexia, hyperthermia, tachycardia, diaphoresis, tachypnoea, and death.

In some embodiments, the adverse events or side effects comprise at least one of pyrexia, hyperthermia, tachycardia, diaphoresis, tachypnoea, and death.

In some embodiments, the adverse event or side effect is characterized by at least one of elevated body temperature, elevated heart rate, abnormal sweating, erythema, perspiration, dehydration, and abnormally rapid breathing.

In some embodiments, the adverse event or side effect is associated with cardiovascular collapse, cardiac arrest, and/or death.

In some embodiments, the adverse event or side effect is associated with cardiac arrest.

In some embodiments, the subject does not experience a significant increase in body temperature or a significant increase in heart rate.

In some embodiments, the subject experiences a saturable absorption of the compound of formula (I) such that overdose is prevented. In some embodiments, there is a saturation of absorption at high single doses. In some embodiments, there is a saturation of absorption at a single oral dose of above 500 mg, above 600 mg, above 700 mg, above 800 mg, above 900 mg, above 1000 mg, above 1050 mg, above 1100 mg, above 1200 mg, above 1300 mg, or above 1400 mg of the compound of formula (I). In some embodiments, the subject does not experience a correlation between dose and toxicity, adverse events, side effects, or overdose. In some embodiments, the clinically significant risk of adverse events, side effects, toxicity, and/or overdoes is prevented by at least one of: i) extending the half-life (t½) of a phenolic metabolite of the compound of formula (I); ii) delaying the time to maximum plasma concentration (Tmax) of a phenolic metabolite; iii) lowering maximum plasma concentration (Cmax) of the phenolic metabolite of the compound of formula (I); and iv) increasing the area under the curve (AUC).

In some embodiments, the clinically significant risk of adverse events, side effects, toxicity, and/or overdoes is prevented by providing a steady state of maximum plasma concentration (Cmax) of the phenolic metabolite of the compound of formula (I) from about 80 ng/ml to about 8300 ng/mL with administration of the compound of formula (I).

In some embodiments, the clinically significant risk of adverse events, side effects, toxicity, and/or overdoes is prevented by providing a mean half-life (t½) of the phenolic metabolite of the compound of formula (I) about 20-50 hours, about 25-40 hours, or about 30-40 hours with administration of the compound of formula (I). [0232] In some embodiments, the clinically significant risk of adverse events, side effects, toxicity, and/or overdoes is prevented by providing a median time to maximum plasma concentration (Tmax) of a phenolic metabolite of the compound of formula (I) enol about 6-8 hours or about 6-10 hours with administration of the compound of formula (I).

In some embodiments, the clinically significant risk of adverse events, side effects, toxicity, and/or overdoes is prevented by providing a median area under the curve extrapolated to infinity (AUCmf) of the phenolic metabolite of the compound of formula (I) about 3 h*μg/mL to about 420 h*μg/iuL with administration of the compound of formula (I).

In some embodiments, the clinically significant risk of adverse events, side effects, toxicity, and/or overdoes is prevented by providing an AUC/Cmax ratio of about 18 with administration of the compound of formula (I).

In some embodiments, the above various methods include providing at least one of i) median time to maximum plasma concentration (Tmax) of the compound of formula (I) about 1-6 hours, about 1-3 hours, or about 1-2 hours; ii) median half-life (t½) of the compound of formula (I) about 1-3 hours or about 1-2 hours; and iii) median area under the curve extrapolated to infinity (AUCmf) of the compound of formula (I) about 18 h*ng/mL to about 380 h*ng/mL. In some embodiments, the subject experiences a reduction in at least one of body weight, blood pressure, and blood glucose after the administration.

In some embodiments, the subject experiences at least one of: i) a reduction of body weight by at least 5% or 10%; ii) a reduction of blood pressure of at least 5 mmHg; iii) a reduction of HbAic by at least 0.5%, or at least 1.5%; iv) a reduction of lipids by at least 10%; v) a reduction of liver fat by at least 50%; vi) a reduction of serum alanine aminotransferase (ALT); and vii) a reduction of aspartate aminotransferase (AST).

In some embodiments, the compound of formula (I) is administered at about 30 mg, 100 mg, 200 mg, 500 mg, or 1050 mg per day.

In certain embodiments, the therapeutically effective amount is from about from about 30 mg to about 1400 mg per day, from about 50 mg to about 100 mg per day, from about 150 mg to about 600 mg per day, or from 200 mg to 550 mg per day.

In certain embodiments, the therapeutically effective amount is about 100 mg, 150 mg, 200 mg, 250 mg, 300 mg, 350 mg, 400 mg, 450 mg, 500 mg, or 600 mg per day.

In certain embodiments, the therapeutically effective amount is about 30 mg, 35 mg, 40 mg, 45 mg, 50 mg, 55 mg, 60 mg, 65 mg, 70 mg, 75 mg, 80 mg, 85 mg, 90 mg, or 95 mg, per day. In certain embodiments, therapeutically effective amount is about 150 mg, 300 mg, or 450 mg per day.

In some embodiments, the therapeutically effective amount is chosen to adjust Cmax, Tmax and AUC.

### Pharmaceutical Salts

the compound of formula I may be used in its native form or as a salt. In cases where forming a stable nontoxic acid or base salt is desired, administration of the compound as a pharmaceutically acceptable salt may be appropriate.

Suitable pharmaceutically acceptable salts include prepared from inorganic and organic acids including sulfate, hydrogen sulfate, hydrochloric, hydrobromic, hydnodic, nitric, carbonic, sulfuric, phosphoric acids, formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxy benzoic, phenylacetic, mandelic, embonic (pamoic), methanesulfonic, ethanesulfonic, benzenesulfonic, pantothenic, trifluoromethanesulfonic, sulfanilic, stearic, alginic, 2-hydroxyethanesulfonic, p-toluene sulfonic, cyclohexylaminosulfonic, salicylic, galactaric, b-hydroxybutyric and galacturonic acid; or prepared from ammonium salts and metallic salts including calcium, magnesium, potassium, sodium and zinc salts. Composition/Formulation

Pharmaceutical compositions of the present disclosure may be manufactured by processes well known in the art, e.g., by means of conventional mixing, dissolving, granulation, drageemaking, levitating, emulsifying, encapsulating, entrapping, lyophilizing processes or spray drying.

Pharmaceutical compositions for use in accordance with the present disclosure may be formulated in conventional manner using one or more pharmaceutically acceptable carriers comprising excipients and auxiliaries, which facilitate processing of the active compound into preparations, which can be used pharmaceutically. Proper formulation is dependent upon the route of administration chosen. Pharmaceutically acceptable excipients and carriers are generally known to those skilled in the art and are thus included in the instant disclosure. Such excipients and carriers are described, for example, in “Remington's Pharmaceutical Sciences” Mack Pub. Co., New Jersey (1991).

In some embodiments, the pharmaceutical composition comprises the compound of formula (I) or a pharmaceutically acceptable salt thereof, and a pharmaceutically acceptable excipient.

In some embodiments, the methods of the present disclosure comprise administering to the subject a pharmaceutical composition comprising the compound of formula (I) or a pharmaceutically acceptable salt thereof, and a pharmaceutically acceptable excipient.

### Pharmaceutical Dosage Forms

The present disclosure includes novel pharmaceutical dosage forms of the compound of formula (I) or a pharmaceutically acceptable salt thereof. The dosage forms described herein are suitable for oral administration to a subject. The dosage form may be in any form suitable for oral administration, including, but not limited to, a capsule or a tablet. In some embodiments, the present disclosure provides a single unit dosage capsule or tablet form containing from about 30 mg to about 1400 mg, from about 100 mg to about 1000 mg, from about 150 mg to about 600 mg, or from 200 mg to 550 mg of the compound of formula (I) or a pharmaceutically acceptable salt thereof. In some embodiments, the compound of formula (I) or a pharmaceutically acceptable salt thereof is administered in a hydroxypropyl methylcellulose capsule.

In some embodiments, the amount of the compound of formula (I) in a unit dosage is about 30 mg, 50 mg, 75 mg, 100 mg, 150 mg, 170 mg, 200 mg, 250 mg, 300 mg, 340 mg, 350 mg, 400 mg, 450 mg, 500 mg, 510 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, or 1400 mg. In some embodiments, the single unit dosage form is a capsule. In some embodiments, the single unit dosage form is a tablet.

In some embodiments, the amount of the compound of formula (I) in a unit dosage is about 30 mg, 40 mg, 50 mg, 60 mg, 70 mg, 75 mg, 80 mg, 90 mg, 100 mg, 150 mg, 170 mg, 200 mg, 250 mg, 300 mg, 340 mg, 350 mg, 400 mg, 450 mg, 500 mg, 510 mg, 550 mg, 600 mg, 650 mg, 700 mg, 750 mg, 800 mg, 850 mg, 900 mg, 950 mg, 1000 mg, 1050 mg, 1 100 mg, 1150 mg, 1200 mg, 1250 mg, 1300 mg, 1350 mg, or 1400 mg. In some embodiments, the single unit dosage form is a capsule.

In some embodiments, the single unit dosage form is a tablet.

In some embodiments, the amount of the compound of formula (I) in a unit dosage is about 30 mg, 100 mg, 200 mg, 500 mg, 600 mg, 1050 mg, or 1400 mg. In some embodiments, the amount of the compound of formula (I) in a unit dosage is about 200 mg, 400 mg, or 550 mg. In some embodiments, the amount of the compound of formula (I) in a unit dosage is about 170 mg, 340 mg, 510 mg. In some embodiments, the amount of the compound of formula (I) in a unit dosage is about 150 mg, 300 mg, 450 mg.

In some embodiments, the amount of the compound of formula (I) in a unit dosage is about 30 mg, 35 mg, 40 mg, 45 mg, 50 mg, 55 mg, 60 mg, 65 mg, 70 mg, 75 mg, 80 mg, 85 mg, 90 mg, or 95 mg, per day. Routes of Administration

In therapeutic use for controlling or preventing weight gain in a mammal, a compound of the present disclosure or its pharmaceutical compositions can be administered orally, or parenterally.

In certain embodiments, the compound of the present disclosure or its pharmaceutical compositions can be administered once daily orally.

As used herein, “pharmaceutically acceptable” means suitable for use in human, companion animals, and livestock animals.

As used herein, the letter “H” refers to a hydrogen atom.

As used herein, the letter “D”, when associate with a variable such as R¹, R², R³, R⁴, or R⁵, refers to a deuterium atom.

As used herein, the term “metabolic disorder” refers to a condition characterized by an alteration or disturbance in metabolic function.

As used herein, the phrase “pharmaceutically acceptable salt” refers to a salt that is pharmaceutically acceptable non-toxic acids, including inorganic acids, organic acids, solvates, or hydrates thereof.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material.

In some embodiments, the compound of formula (I) or a pharmaceutically acceptable salt thereof of the present disclosure is useful for treating mitochondria-related disorders, including, but not limited to, obesity, diabetes, insulin resistance, and heart or renal failure in a subject in need thereof.

In some embodiments, the compound of formula (I) or a pharmaceutically acceptable salt thereof of the present disclosure is useful for treating disease, disorders, and conditions which are associated with defects in mitochondrial function in a mammal in need thereof.

In some embodiments, formula (I) has the following structure:

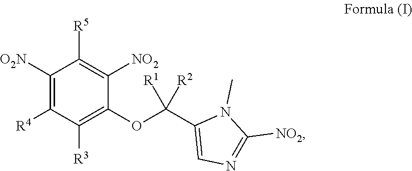

or a pharmaceutically acceptable salt thereof, wherein R¹-R⁵ independently are H or D provided that at least one of R¹-R⁵ is D.

In some embodiments, R¹ and R² are D; R³, R⁴, and R⁵ independently are H or D.

In some embodiments, R¹ is H; R² is D; R³, R⁴, and R⁵ independently are H or D.

In some embodiments, R¹ and R² are D; R³, R⁴, and R⁵ are H (Compound A).

In some embodiments, R¹ is H; R² is D; R³, R⁴, and R⁵ are H (Compound B).

In some embodiments, R¹, R², R³, R⁴, and R⁵ are D (Compound C).

In some embodiments, R¹ and R² are H; R³, R⁴, and R⁵ are D (Compound X).

In some embodiments, R¹ is H; R² is D; R³, R⁴, and R⁵ are D (Compound Y).

In some embodiments, R¹ and R² are H; at least one of R³, R⁴, or R⁵ is D.

In some embodiments, R¹ is H; R² is D; at least one of R³, R⁴, or R⁵ is D

In some embodiments, the compound of formula (I) or a pharmaceutically acceptable salt thereof of the present disclosure can stimulate oxygen consumption rate (OCR) in a subject in need thereof.

In some embodiments, the compound of formula (I) or a pharmaceutically acceptable salt thereof of the present disclosure is useful for treating diabetes, including but not limiting, non-alcoholic fatty liver disease (NAFLD), non-alcoholic steatohepatitis (NASH), hepatic steatosis, and type 2 diabetes (T2DM) in a subject in need thereof.

In some embodiments, the compound of formula (I) or a pharmaceutically acceptable salt thereof of the present disclosure is useful for treating obesity or reducing adiposity in a subject in need thereof.

In some embodiments, the compound of formula (I) or a pharmaceutically acceptable salt thereof of the present disclosure is useful for controlling or preventing from weight gain or maintaining of a weight in a subject in need thereof.

In some embodiments, the compound of formula (I) or a pharmaceutically acceptable salt thereof of the present disclosure is useful for controlling or preventing obesity or excess body fat in a subject in need thereof.

In some embodiments, the compound of formula (I) or a pharmaceutically acceptable salt thereof of the present disclosure is useful for treating cardiovascular disease in a subject in need thereof.

In some embodiments, the compound of formula (I) or a pharmaceutically acceptable salt thereof of the present disclosure is useful for treating inflammation and fibrosis resulting in NASH.

In another aspect, disclosed herein is a deuterium-enriched 5-[(2,4-dinitrophenoxy)methyl]-1-methyl-2-nitro-1H-imidazole (DNP)

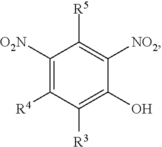

wherein R³, R⁴, R⁵ is H or D provided that at least one of them is D.

### Routes of Administration

In therapeutic use for controlling or preventing weight gain in a subject, the compound of formula (A) or a pharmaceutically acceptable salt thereof of the present disclosure can be administered orally or parenterally.

In certain embodiments, the compound of the present disclosure or its pharmaceutical compositions can be administered once, twice, or three times daily.

### Composition/Formulation

In some embodiments, the present disclosure provides a pharmaceutical composition comprising a pharmaceutically acceptable carrier and the compound of formula (I) or a pharmaceutically acceptable salt thereof.

Pharmaceutical compositions for use in accordance with the present disclosure may be formulated in conventional manner using one or more pharmaceutically acceptable carriers comprising excipients and auxiliaries, which facilitate processing of the active compound into preparations that can be used pharmaceutically. Proper formulation is dependent upon the route of administration chosen. Pharmaceutically acceptable excipients and carriers are generally known to those skilled in the art. Such excipients and carriers are described, for example, Martin, Remington's Pharmaceutical Sciences, 15th Ed., Mack Publ. Co., Easton, PA (1975) or Rowe, Shesky, and Quinn, Handbook of Pharmaceutical Excipients, 6^{th} Ed. Pharmaceutical Press, London, UK (2009)).

### Dosage

Pharmaceutical compositions suitable for use in the present disclosure include compositions wherein the compound of formula (I) are contained in an amount sufficient to achieve the intended purpose.

Determination of a therapeutically effective amount is well within the capability of those skilled in the art. Generally, the quantity of the compound of formula (I) or a pharmaceutically acceptable salt will range between 0.01% and 99.9% by weight of the composition. In some embodiments, the quantity of the compound of formula (I) or a pharmaceutically acceptable salt will range between 0, 1% and 90% by weight of the composition. In some embodiments, the quantity of the compound of formula (I) or a pharmaceutically acceptable salt will range between 1% and 70% by weight of the composition. In some embodiments, the quantity of the compound of formula (I) or a pharmaceutically acceptable salt will range between 10% and 50% by weight of the composition.

A therapeutically effective amount of the compound of formula (I) or a pharmaceutically acceptable salt is in the range of about 0.001 to about 1000 mg/kg of body weight/day. The desired dose may conveniently be presented in a single dose or divided doses administered at appropriate intervals, for example, as two, three, four or more sub-doses per day.

In some embodiments, the effective amount of the compound of formula (I) or a pharmaceutically acceptable salt is about 0.01 mg/kg to about 100 mg/kg.

In some embodiments, the effective amount of the compound of formula (I) or a pharmaceutically acceptable salt is between about 0.1 mg/kg to about 50 mg/kg and any and all whole or partial increments there between. For example, including but not limiting, about 0.1 mg/kg, about 1 mg/kg, about 10 mg/kg, about 100 mg/kg, about 200 mg/kg, or about 300 mg/kg.

In some embodiments, the effective amount of the compound of formula (I) or a pharmaceutically acceptable salt is about 1-10 mg/kg. In some embodiments, the effective amount of the compound of formula (I) or a pharmaceutically acceptable salt is about 2-10 mg/kg. In other embodiments, the effective amount of the compound of formula (I) or a pharmaceutically acceptable salt is about 3-10 mg/kg. In other embodiments, the effective amount of the compound of formula (I) or a pharmaceutically acceptable salt is about 4-10 mg/kg.

### Pharmaceutical Salts

The compound of formula (I) may be used in its native form or as a salt. In cases where forming a stable nontoxic acid or base salt is desired, administration of the compound as a pharmaceutically acceptable salt may be appropriate.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxy benzoic, phenylacetic, mandelic, embonic (pamoic), methanesulfonic, ethanesulfonic, benzenesulfonic, pantothenic, trifluoromethanesulfonic, sulfanilic, stearic, alginic, 2-hydroxyethanesulfonic, p-toluene sulfonic, cyclohexylaminosulfonic, salicylic, galactaric, β-hydroxybutyric and galacturonic acid.

### EXAMPLES

### Example 1 Compound (A) Effects on Oxygen Consumption Rate (OCR)

HepG2 (hepatocellular carcinoma cells) were utilized in a Seahorse XFe96 Extracellular Flux Analyzer to analyze Compound (A)'s and the phenolic metabolite of the compound of formula (I)'s (“DNP”) effect on oxygen consumption rate (OCR).

30,000 HepG2 cells in MEM Alpha growth medium+10% Fetal Bovine Serum were seeded in XFe 96-well cell culture plate and incubated overnight at 37 °C with 5% CO2. The following day, cells were transferred to assay media (DMEM basal assay medium containing 10 mM Glucose 2 mM Glutamine, 1 mM Sodium Pyruvate, adjusted to pH7.4 with IN NaOH) and OCR was measured in a Seahorse XFe96 Extracellular Flux Analyzer according to the manufacturer's instructions. The effect of novel compounds on OCR was assayed in quadruplicates, at three different concentrations and in three separate experiments. The concentration that reaches half their maximum effect (EC₅₀) and maximum OCR (Emax) increase as compared to DNP (100%) over untreated cells (0%) was calculated as an average between the three independent experiments. For data analysis GraphPad Prism was utilized.

### Results:

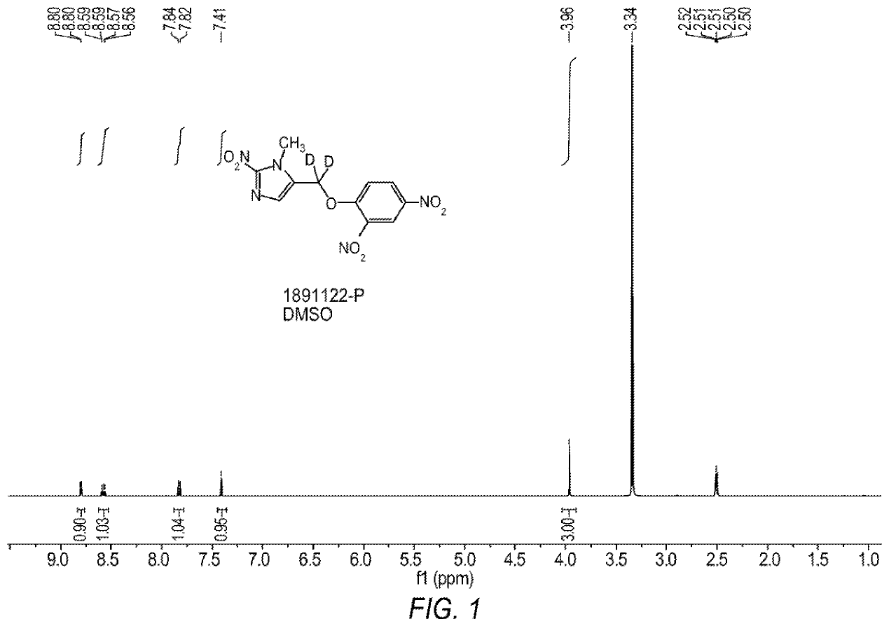

DNP and Compound (A) increased oxygen consumption rate (OCR). The OCR values were normalized to 100 μM DNP (Emax=100%) and calculated Emax for deuterated DNP is 82.61%. FIG. 1 shows that the EC50 for deuterated DNP and DNP are 35 μM and 22 μM 2respectively. Methanol concentration in samples has no effect on assay performance.

Summary: Compound (A) is activated by CYP enzymes in a similar manner and at similar rates as compared to the corresponding non-deuterated compound, i.e. R¹-R⁵ are H in the compound of formula (I).

### Example 2 General Synthetic Scheme

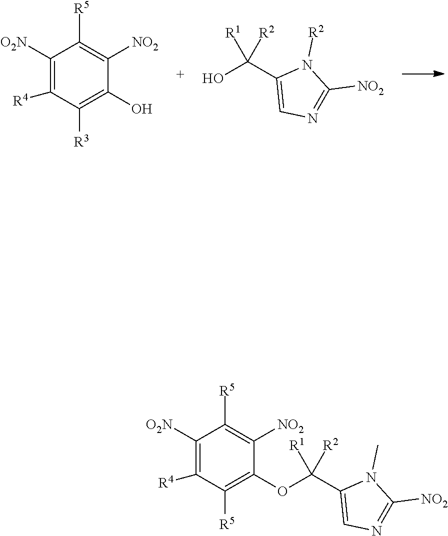

wherein R¹ is H; R² is D; R³, R⁴, and R⁵ independently are H or D provided that at least one of R¹-R⁵ is D.

The compounds of Formula (I) can be produced by synthetic procedures known to those having skill in the art, for example, the procedures disclosed in WO 2018/129258. As provided in the scheme, Mitsunobu chemistry can be used to activate the hydroxyl oxygen of the imidazole compound using a reagent combination such as diisopropyl azodicarboxylate (DIAD) or diethyl azodicarboxylate (DEAD) with triphenylphosphine, which gives way to nucleophilic substitution of the activated hydroxyl with phenol.

### Example 3 Preparation of Compound (A)

### Step 1: Preparation of (1-methyl-2-nitro-1H-imidazol-5-yl) methan-d₂-ol

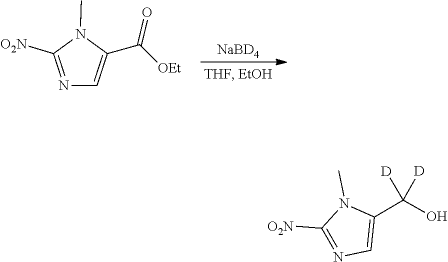

### Step 2: Preparation of Compound (A)

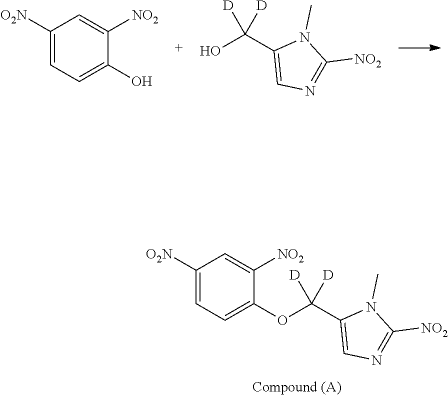 [SMILES: O=[N+]([O-])c1ccc(O)c([N+](=O)[O-])c1 ; [2H]C([2H])(O)c1cnc([N+](=O)[O-])n1C ; [2H]C([2H])(Oc1ccc([N+](=O)[O-])cc1[N+](=O)[O-])c1cnc([N+](=O)[O-])n1C]

Following the procedure described in U.S. Pat. No. 8,003,625 B2, 2,4-dinitrophenol (wetted with ca. 20% water, from TCI America, Cat. No. D0109) (269 mg wet weight, 215 mg dry weight, 1.17 mmol) is dissolved in methylene chloride (2 mL) and stirred with anhydrous sodium sulfate at room temperature for 3 hours. The methylene chloride solution is decanted into a reaction flask and the sodium sulfate is washed with additional methylene chloride (2 mL). To the solution is added (1-(dideuteromethyl)-2-nitro-1H-imidazol-5-yl) methan-d₂-ol (115 mg, 0.732 mmol) and triphenylphosphine (211 mg, 0.805 mmol). The mixture is stirred at room temperature until a solution is achieved. The solution is then cooled in an ice bath and treated with diisopropyl azodicarboxylate, DIAD (158 μL, 0.805 mmol). After 1 hour the ice bath is removed and the mixture is stirred overnight at room temperature. Crude product is purified on a silica gel column to isolate the product mixed with triphenylphosphine oxide. The solids are triturated with t-butyl methyl ether to remove the triphenylphosphine oxide to afford 5-[(2,4-dinitrophenoxy)methyl]-1-methyl-2-nitro-1H-imidazole (70 mg, 0.236 mmol, 30% yield).

¹H NMR (DMSO) δ 8.80, 8.59, 8.57, 8.56, 3.96, 3.34, 2.52, 2.51, 2.50 as shown below:

We claim:

1. A compound having the following structure: 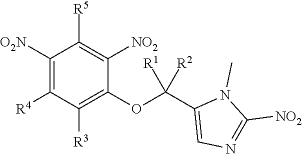
or a pharmaceutically acceptable salt thereof, wherein R¹-R⁵ independently are H or D provided that at least one of R¹-R⁵ is D.

2. The compound of claim 1, wherein R¹ and R² are D; R³, R⁴, and R⁵ are H.

3. The compound of claim 1, wherein R¹ is H; R² is D; R³, R⁴, and R⁵ are H.

4. The compound of claim 1, wherein R¹, R², R³, R⁴, and R⁵ are D.

5. The compound of claim 1, wherein R¹ and R² are H; R³, R⁴, and R⁵ are D.

6. The compound of claim 1, wherein R¹ is H; R² is D; R³, R⁴, and R⁵ are D.

7. The compound of claim 1, wherein R¹ and R² are D; R³, R⁴, and R⁵ independently are H or D.

8. The compound of claim 1, wherein R¹ is H; R² is D; R³, R⁴, and R⁵ independently are H or D.

9. The compound of claim 1, wherein R¹ and R² are H; at least one of R³, R⁴, or R⁵ is D.

10. The compound of claim 1, wherein R¹ is H; R² is D; at least one of R³, R⁴, or R⁵ is D.

11. A method of treating mitochondria-related disorders or conditions in a subject comprising administering to the subject in need thereof an effective amount of the compound of any one of claims 1-10, or a pharmaceutically acceptable salt thereof.

12. The method of claim 11, wherein the disorder or condition is metabolic disorders, diabetes, or diabetes-associated complications.

13. The method of claim 12, wherein the disorder is obesity or excess body fat.

14. The method of claim 12, wherein the disorder is diabetes.

15. The method of claim 12, wherein the disorder is type 2 diabetes (T2DM).

16. The method of claim 12, wherein the disorder is non-alcoholic fatty liver disease (NAFLD).

17. The method of claim 12, wherein the disorder is non-alcoholic steatohepatitis (NASH).

18. The method of claim 12, wherein the disorder is hepatic steatosis.

19. The method of claim 12, wherein the disorder is insulin resistance or intolerance.

20. The method of claim 12, wherein the disorder is dyslipidemia.

21. The method of claim 12, wherein the disorder is cardiovascular disease.

22. The method of claim 12, wherein the disorder is atherosclerosis.

23. A method of reducing adiposity, controlling or preventing of weight gain in a subject comprising administering to the subject in need thereof an effective amount of the compound of any one of claims 1-10 or a pharmaceutically acceptable salt thereof.

24. A method for stimulating oxygen consumption rate (OCR) in a subject comprising administering to the subject in need thereof an effective amount of the compound of any one of claims 1-10 or a pharmaceutically acceptable salt thereof.

25. A method for treating inflammation and fibrosis resulting in NASH in a subject comprising administering to the subject in need thereof an effective amount of the compound of any one of claims 1-10 or a pharmaceutically acceptable salt thereof.

26. A method of reducing a cardiovascular risk or mortality in a subject suffering from a symptom due to a cardiovascular disease, comprising administering to the subject a therapeutically effective amount of the compound of claim 1, or a pharmaceutically acceptable salt thereof.

27. The method of claim 26, wherein the symptom is shortness of breath, shortness of breath with exertion, dizziness, chest pain, syncope, fatigue, impaired energetics in the heart, or limits on activities of daily living.

28. The method of claim 26, wherein the limit on an activity of daily living is difficulties on personal care, mobility, or eating.

29. The method of claim 26, wherein the cardiovascular disease comprises heart failure, heart attack, coronary artery disease, or coronary heart disease (CHD).

30. The method of claim 26, wherein heart failure comprises heart failure with preserved ejection fraction (HFpEF), heart failure with reduced ejection fraction (HFrEF), or heart failure with mid-range ejection fraction (HFmrEF).

31. The method of any one of claims 26-30, wherein the subject experiences a reduction in the risk of a major cardiovascular event after administration.

32. The method of claim 31, wherein the major cardiovascular event is death or hospitalization for worsening of the disease.

33. A method for treating HFpEF, HFrEF, or HFmrEF in a subject, comprising administering to the subject a therapeutically effective amount of the compound of claim 1 or a pharmaceutically acceptable salt thereof.

34. The method of claim 33, wherein the subject is suffering from at least one of the symptoms selected from shortness of breath, shortness of breath with exertion, impaired energetics in the heart, dizziness, fatigue, dyspnea, palpitations (atrial fibrillation), chest discomfort, edema, syncope, and a limit on an activity of daily living.

34. The method of claim 33, wherein the limit on an activity of daily living is difficulties on personal care, mobility, and eating.

35. The method of claim 33, wherein the subject is suffering from at least one of symptoms selected from reduced exercise tolerance, fatigue, tiredness, increased time to recover after exercise, and ankle swelling.

34. The method of claim 33, wherein the subject is suffering from at least one of symptoms selected from coronary artery disease, hypertension, and heart murmur.

35. The method of claim 26 or 33, wherein the subject experiences an improvement of cardiac bioenergetic deficiency after administration, wherein the improvement comprising: a) weight loss >5%, b) reduction in blood pressure, and/or c) a reduction in the risk of a major cardiovascular event, wherein the major cardiovascular event is selected from the group consisting of death, hospitalization for worsening of the disease, and myocardial infraction.

36. The method of claim 26 or 33, further comprising assessing peak oxygen consumption (VO2) and/or VE/CO2 or VE/VCO2 slope in the subject during exercise before and after administration of the therapeutically effective amount of the compound of formula (I) wherein an increase in VO2 in the subject after administration indicates a reduction in the extent of HFpEF, HFrEF, HFmrEF or one or more symptomatic components or conditions of cardiovascular disease thereof in the subject.

37. The method of claim 26 or 33, wherein the method increases VO2 in the subject after administration.

38. The method of claim 26 or 33, further comprising assessing 6-minute walk distance (6MWD) in the subject during exercise before and after administration of the therapeutically effective amount of 5-[(2,4-dinitrophenoxy)methyl]-1-methyl-2-nitro-1H-imidazole, wherein an increase in 6MWD in the subject after administration indicates a reduction in the extent of HFpEF or the at least one symptomatic component or condition thereof in the subject.

39. The method of claim 26 or 33, wherein the method increases 6MWD after the administration.

40. The method of claim 26 or 33, wherein the treatment further comprises assessing an NYHA classification score of the subject before and after administration.

41. The method of claim 40, wherein a decreased NYHA score after administration indicates a reduction in the extent of the cardiac disease in the subject.

42. The method of claim 40, wherein the method decreases the NYHA classification score of the subject after administration from Class III to Class II, or from Class II to Class I.

43. A method of reducing blood pressure in a subject, comprising administering to the subject a therapeutically effective amount of the compound of formula (I) of claim 1, or a pharmaceutically acceptable salt thereof.

44. The method of claim 43, wherein the subject is suffering from cardiovascular disease, hypertension, resistant hypertension, or severe hypertension.

45. The method of claim 44, wherein the cardiovascular disease comprises heart failure, heart attack, coronary artery disease, or coronary heart disease (CHD).

46. The method of claim 45, wherein heart failure comprises HFpEF, HFrEF, or HFmrEF.

47. The method of any one of claims 43-46, wherein the subject has hypertension associated with HFpEF, HFrEF, or HFmrEF.

48. The method of any one of claims 43-46, wherein the subject is suffering from at least one of symptoms selected from headaches, shortness of breath, chest pain, nosebleeds, dizziness, fatigue, vision problem, irregular heartbeat, blood in urine, sweating, trouble sleeping, and blood spots in eyes.

49. The method of any one of claims 43-46, wherein the reducing blood pressure comprises reducing diastolic blood pressure and/or reducing systolic blood pressure.

50. The method of claim 43, wherein the subject experiences a reduction of blood pressure of at least 5 mmHg after administration.

51. The method of claim 43, wherein the method reduces or slows the progression of the risk of developing a cardiovascular disease or heart failure.

52. The method of claim 51, wherein heart failure comprises HFpEF, HFrEF, or HFmrEF.

53. The method of any one of claim 26, 33, or 43, wherein the subject suffers from obesity, excess body fat, diabetes, high blood pressure (hypertension), dyslipidemia, hypertriglyceridemia, acquired lipodystrophy, inherited lipodystrophy, partial lipodystrophy, or metabolic syndrome.

54. A method of treating a cardiovascular disease comprising administering a therapeutically effective amount of the compound of formula (I) of claim 1 or a pharmaceutically acceptable salt thereof, in a subject, to achieve at least one of: i) a steady state of maximum plasma concentration (Cmax) of a phenolic metabolite (“metabolite”) of the compound of formula (I) from about 80 ng/ml to about 8300 ng/ml; ii) mean half-life (t½) of the metabolite of about 20-50 hours, about 25-40 hours, or about 30-40 hours; iii) median time to maximum plasma concentration (Tmax) of the metabolite of about 6-8 hours or about 6-10 hours; iv) median area under the curve extrapolated to infinity (AUCinf) of the metabolite of about 3 h*μg/mL to about 420 h*μg/mL; and v) AUC/Cmax ratio of about 18.

55. The method of any one of claim 26, 33, 43, or 54, wherein the method does not cause a clinically significant risk of adverse events after administration.

56. The method of claim 55, wherein the adverse events are selected from the group consisting of nausea, vomiting, sweating, dizziness, headaches, cataracts, glaucoma, pyrexia, hyperthermia, tachycardia, diaphoresis, tachypnoea, and death.

57. The method of claim 56, wherein the adverse event is characterized by at least one of elevated body temperature, elevated heart rate, abnormal sweating, erythema, perspiration, dehydration, and abnormally rapid breathing.

58. A method of treating mitochondria-related disorders or conditions without causing a clinically significant risk of adverse events in a subject, the method comprising administering to the subject a therapeutically effective amount of the compound of claim 1, or a pharmaceutically acceptable salt thereof.

59. The method of claim 58, wherein the disorder is obesity, excess body fat, diabetes, insulin resistance or intolerance, high blood pressure, dyslipidemia, cardiovascular disease, atherosclerosis, hypertriglyceridemia, acquired lipodystrophy, inherited lipodystrophy, partial lipodystrophy, metabolic syndrome, Rett's syndrome, metabolic syndrome associated with aging, metabolic diseases associated with increased reactive oxygen species (ROS), Friedreich's ataxia, or liver disease.

60. The method of claim 59, wherein the diabetes is type 2 diabetes (T2DM).

61. The method of claim 59, wherein the cardiovascular disease comprises heart failure, HFpEF, HFrEF, HFmrEF, heart attack, coronary artery disease, or CHD.

62. The method of claim 59, wherein the liver disease comprises non-alcoholic fatty liver disease (NAFLD), non-alcoholic steatohepatitis (NASH), noncirrhotic NASH, noncirrhotic NASH with liver fibrosis, hepatic steatosis, hepatic fibrosis, liver cirrhosis, or hepatocellular carcinoma.

63. The method of claim 59, wherein the condition is at least on of steatosis, inflammation, fibrosis, cirrhosis, and hepatocyte injury in NASH.

64. The method of any one of claims 59-63, wherein the method provides at least one of the following in the subject after administration: i) a steady state of maximum plasma concentration (Cmax) of a phenolic metabolite (“metabolite”) from about 80 ng/ml to about 8300 ng/ml; ii) mean half-life (t½) of the phenolic metabolite of about 20-50 hours, about 25-40 hours, or about 30-40 hours; iii) median time to maximum plasma concentration (Tmax) of the metabolite of about 6-8 hours or about 6-10 hours; iv) median area under the curve extrapolated to infinity (AUCmf) of the metabolite of about 3 h*μg/mL to about 420 h*μg/mL; and v) AUC/Cmax ratio of about 18.

65. The method of claim 58, wherein the adverse events are associated with a mitochondria uncoupler.

66. The method of claim 65, wherein the mitochondria uncoupler is a metabolite of the compound of formula (I) of claim 1.

67. The method of any one of claims 58-66, wherein the adverse events comprise at least one of nausea, vomiting, sweating, dizziness, headaches, cataracts, glaucoma, pyrexia, hyperthermia, tachycardia, diaphoresis, tachypnoea, and death.

68. The method of claim 67, wherein the adverse event is characterized by at least one of elevated body temperature, elevated heart rate, abnormal sweating, erythema, perspiration, dehydration, and abnormally rapid breathing.

69. The method of claim 67, wherein the adverse event is associated with cardiovascular collapse, cardiac arrest, and/or death.

70. The method of claim 69, wherein the adverse event is associated with cardiac arrest.

71. A method of reducing toxicity or side effects in treating mitochondria-related disorders or conditions in a subject, comprising administering to the subject a therapeutically effective amount of the compound of claim 1 or a pharmaceutically acceptable salt thereof.

72. The method of claim 71, wherein the disorder is disorder is obesity, excess body fat, diabetes, insulin resistance or intolerance, high blood pressure, dyslipidemia, cardiovascular diseases, atherosclerosis, hypertriglyceridemia, acquired lipodystrophy, inherited lipodystrophy, partial lipodystrophy, metabolic syndrome, Rett's syndrome, metabolic syndrome associated with aging, metabolic diseases associated with increased reactive oxygen species (ROS), Friedreich's ataxia, or liver diseases.

73. The method of claim 72, wherein the diabetes is type 2 diabetes (T2DM).

74. The method of claim 72, wherein the cardiovascular diseases comprise heart failure, HFpEF, HFrEF, HFmrEF, heart attack, coronary artery disease, or CHD.

75. The method of claim 72, wherein the liver diseases comprise non-alcoholic fatty liver disease (NAFLD), non-alcoholic steatohepatitis (NASH), noncirrhotic NASH, noncirrhotic NASH with liver fibrosis, hepatic steatosis, hepatic fibrosis, liver cirrhosis, or hepatocellular carcinoma.

76. The method of claim 71, wherein the condition is at least one of steatosis, inflammation, fibrosis, cirrhosis, and hepatocyte injury in NASH.

77. The method of claim 71, wherein the side effects are associated with a mitochondria uncoupler.

78. The method of claim 77, wherein the mitochondria uncoupler is a phenolic metabolite of the compound of formula (I) of claim 1.

79. The method of claim 78, comprising at least one of: i) extending the half-life (t½) of the phenolic metabolite of the compound of claim 1; ii) delaying the time to maximum plasma concentration (Tmax) of of the phenolic metabolite of the compound of claim 1; iii) lowering maximum plasma concentration (Cmax) of the phenolic metabolite of the compound of claim 1; and iv) increasing the area under the curve (AUC).

80. The method of claim 79, wherein the mean half-life is extended to about 20-50 hours, 25-40 hours, or 30-40 hours.

81. The method of claim 79, wherein median Tmax is extended to at least 6 hours or at least 8 hours.

83. The method of claim 79, wherein median Tmax is extended to about 6-8 hours or about 6-10 hours.

84. The method of claim 79, wherein lowering the metabolite Cmax comprises providing a steady state of Cmax of the metabolite from about 80 ng/ml to about 8300 ng/ml in the subject after administration.

85. The method of claim 79, wherein the method provides an AUC/Cmax ratio of about 18 in the subject.

86. The method of any one of claims 71-85, wherein the side effects comprise at least one of nausea, vomiting, sweating, dizziness, headaches, cataracts, glaucoma, pyrexia, hyperthermia, tachycardia, diaphoresis, tachypnea, and death.

87. The method of claim 86, wherein the side effect is characterized by at least one of elevated body temperature, elevated heart rate, abnormal sweating, erythema, perspiration, dehydration, and abnormally rapid breathing.

88. The method of claim 87, wherein the side effect is associated with cardiovascular collapse, cardiac arrest, and/or death.

89. The method of claim 88, wherein the side effect is associated with cardiac arrest.

90. A method of preventing overdose in treating mitochondria-related disorders or conditions in a subject, comprising administering to the subject a therapeutically effective amount of the compound of formula 1 of claim 1, or a pharmaceutically acceptable salt thereof.

91. The method of claim 90, wherein the disorder is obesity, excess body fat, diabetes, insulin resistance or intolerance, high blood pressure, dyslipidemia, cardiovascular diseases, atherosclerosis, hypertriglyceridemia, acquired lipodystrophy, inherited lipodystrophy, partial lipodystrophy, metabolic syndrome, Rett's syndrome, metabolic syndrome associated with aging, metabolic diseases associated with increased reactive oxygen species (ROS), Friedreich's ataxia, or liver diseases.

92. The method of claim 91, wherein the diabetes is type 2 diabetes (T2DM).

93. The method of claim 91, wherein the cardiovascular diseases comprise heart failure, HFpEF, HFrEF, HFmrEF, heart attack, coronary artery disease, or CHD.

94. The method of claim 91, wherein the liver diseases comprise non-alcoholic fatty liver disease (NAFLD), non-alcoholic steatohepatitis (NASH), noncirrhotic NASH, noncirrhotic NASH with liver fibrosis, hepatic steatosis, hepatic fibrosis, liver cirrhosis, or hepatocellular carcinoma.

95. The method of claim 94, wherein the condition is at least on of steatosis, inflammation, fibrosis, cirrhosis, and hepatocyte injury in NASH.

96. The method of claim 90, wherein the overdose is associated with a mitochondria uncoupler.

97. The method of claim 96, wherein the mitochondria uncoupler is a phenolic metabolite of the compound of formula (I) of claim 1.

98. The method of claim 97, wherein the method provides at least one of the following in the subject after administration: i) a steady state of maximum plasma concentration (Cmax) of the metabolite of from about 80 ng/mL to about 8300 ng/ml; ii) mean half-life (t½) of the phenolic metabolite of about 20-50 hours, about 25-40 hours, or about 30-40 hours; iii) median time to maximum plasma concentration (Tmax) of the phenolic metabolite of about 6-8 hours or about 6-10 hours; iv) median area under the curve extrapolated to infinity (AUCinf) of the metabolite of about 3 h*μg/mL to about 420 h*μg/mL; and v) AUC/Cmax ratio of about 18.

99. The method of any one of claims 90-98, wherein the method does not cause a clinically significant risk of adverse events.

100. The method of claim 90, wherein the adverse events comprise at least one of nausea, vomiting, sweating, dizziness, headaches, cataracts, glaucoma, pyrexia, hyperthermia, tachycardia, diaphoresis, tachypnoea, and death.

101. The method of claim 90, wherein the adverse event is characterized by at least one of elevated body temperature, elevated heart rate, abnormal sweating, erythema, perspiration, dehydration, and abnormally rapid breathing.

102. The method of claim 90, wherein the adverse event is associated with cardiovascular collapse, cardiac arrest, and/or death.

103. The method of claim 90, wherein the adverse event is associated with cardiac arrest.

104. A method for increasing metabolic rate without causing a clinically significant risk of adverse events in a subject, comprising administering to the subject a therapeutically effective amount of the compound of formula (I) of claim 1, or a pharmaceutically acceptable salt thereof.

105. The method of claim 104, wherein the subject suffers from at least one of obesity, excess body fat, type 2 diabetes, insulin resistance or intolerance, high blood pressure, dyslipidemia, atherosclerosis, hypertriglyceridemia, acquired lipodystrophy, inherited lipodystrophy, partial lipodystrophy, metabolic syndrome, Rett's syndrome, metabolic syndrome associated with aging, metabolic diseases associated with increased reactive oxygen species (ROS), Friedreich's ataxia, NAFLD, NASH, noncirrhotic NASH, noncirrhotic NASH with liver fibrosis, hepatic steatosis, hepatic fibrosis, liver cirrhosis, and hepatocellular carcinoma.

106. The method of claim 104 or 105, comprising increasing resting metabolic rate without causing a clinically significant risk of adverse events.

107. The method of claim 106, wherein the resting metabolic rate is increased by at least 10%.

108. The method of claim 106, wherein the resting metabolic rate is increased by at least 20%.

109. A method for increasing resting energy expenditure in a subject comprising administering to the subject a therapeutically effective amount of the compound of formula (I) of claim 1, or a pharmaceutically acceptable salt thereof.

110. The method of claim 109, wherein the subject suffers from at least one of obesity, excess body fat, type 2 diabetes, insulin resistance or intolerance, high blood pressure, dyslipidemia, atherosclerosis, hypertriglyceridemia, acquired lipodystrophy, inherited lipodystrophy, partial lipodystrophy, metabolic syndrome, Rett's syndrome, metabolic syndrome associated with aging, metabolic diseases associated with increased reactive oxygen species (ROS), Friedreich's ataxia, NAFLD, NASH, noncirrhotic NASH, noncirrhotic NASH with liver fibrosis, hepatic steatosis, hepatic fibrosis, liver cirrhosis, and hepatocellular carcinoma.

111. The method of any one of claims 104-110, wherein the subject experiences an increase of resting energy expenditure of at least 10% after the administration.

112. The method of any one of claims 104-110, wherein the subject experiences an increase of resting energy expenditure of at least 20% after the administration.

113. The method of any one of claims 104-110, wherein the subject experiences an increase of resting energy expenditure of about 30% after the administration.

114. A method for treating dysmetabolism in a subject comprising administering to the subject a therapeutically effective amount of the compound of formula (I) of claim 1, or a pharmaceutically acceptable salt thereof.

115. The method of claim 114, wherein the subject suffers from at least one of obesity, excess body fat, type 2 diabetes, insulin resistance or intolerance, high blood pressure, dyslipidemia, atherosclerosis, hypertriglyceridemia, acquired lipodystrophy, inherited lipodystrophy, partial lipodystrophy, metabolic syndrome, Rett's syndrome, metabolic syndrome associated with aging, metabolic diseases associated with increased reactive oxygen species (ROS), Friedreich's ataxia, NAFLD, NASH, noncirrhotic NASH, noncirrhotic NASH with liver fibrosis, hepatic steatosis, hepatic fibrosis, liver cirrhosis, and hepatocellular carcinoma.

116. The method of claim 114, wherein the subject suffers from at least one of: increased blood pressure, high blood sugar, excess body fat around the waist, and abnormal cholesterol or triglyceride levels.

117. The method of any one of claims 114-116, wherein the method does not cause a clinically significant risk of adverse events in the subject after administration.

118. The method of any one of claims 114-117, wherein the adverse events are associated with a mitochondria uncoupler.

119. The method of claim 118, wherein the mitochondria uncoupler is a phenolic metabolite of the compound of formula (I) of claim 1.

120. The method of claim 119, wherein the method provides at least one of the following in the subject after administration: i) a steady state of maximum plasma concentration (Cmax) the phenolic metabolite from about 80 ng/ml to about 8300 ng/ml; ii) mean half-life (t½) of phenolic metabolite of about 20-50 hours, about 25-40 hours, or about 30-40 hours; iii) median time to maximum plasma concentration (Tmax) of the phenolic metabolite of about 6-8 hours or about 6-10 hours; iv) median area under the curve extrapolated to infinity (AUCinf) of the phenolic metabolite of about 3 h*μg/mL to about 420 h*μg/mL; and v) AUC/Cmax ratio of about 18.

121. The method of any one of claims 117-120, wherein the adverse events comprise at least one of nausea, vomiting, sweating, dizziness, headaches, cataracts, glaucoma, pyrexia, hyperthermia, tachycardia, diaphoresis, tachypnoea, and death.

122. The method of claim 121, wherein the adverse event is characterized by at least one of elevated body temperature, elevated heart rate, abnormal sweating, erythema, perspiration, dehydration, and abnormally rapid breathing.

123. The method of claim 122, wherein the adverse event is associated with cardiovascular collapse, cardiac arrest, and/or death.

124. The method of claim 123, wherein the method results in a cardiovascular adverse event in less than 10% of patients during the 30 days treatment period.

125. The method of any one of claims 117 to 124 further comprising the step of determining at least one of: homeostatic model assessment for insulin resistance (HOMA-IR); fasting blood glucose concentrations; glycated albumin concentrations; glycosylated hemoglobin (hemoglobin Ale, HbAic) of the subject before and after administration of the therapeutically effective amount of the compound of formula (I) according to claim 1.

126. The method of claim 125, wherein the subject experiences a reduction in at least one of body weight, blood pressure, and blood glucose after the administration.

127. The method of claim 126, wherein the subject experiences at least one of: i) a reduction of body weight by at least 5% or at least 10%; ii) a reduction of blood pressure of at least 5 mmHg; iii) a reduction of HbAu by at least 0.5%, or at least 1.5%; iv) a reduction of lipids by at least 10%; and v) a reduction of liver fat by at least 50%.

128. The method of any one of claims 104-127, wherein the method slows the progression of at least one of: atherosclerosis, NAFLD, NASH, noncirrhotic NASH, noncirrhotic NASH with liver fibrosis, hepatic steatosis, hepatic fibrosis, liver cirrhosis, and hepatocellular carcinoma.

129. The method of any one of claims 104-128, wherein the method accelerates human body's natural processes to improve cardio-metabolic processes.

130. A method of treating hypertriglyceridemia associated with cardiovascular disease, atherosclerosis, obesity, hypertension, diabetes, insulin resistance, and/or liver disease in a subject, comprising administering to the subject a therapeutically effective amount of a compound of formula (I) of claim 1 or a pharmaceutically acceptable salt thereof.

131. The method of claim 130, wherein the cardiovascular disease comprises heart failure, HFpEF, HFrEF, HFmrEF, heart attack, coronary artery disease, or CHD.

132. The method of claim 130, wherein the liver diseases comprise non-alcoholic fatty liver disease (NAFLD), non-alcoholic steatohepatitis (NASH), noncirrhotic NASH, noncirrhotic NASH with liver fibrosis, hepatic steatosis, hepatic fibrosis, liver cirrhosis, or hepatocellular carcinoma.

133. The method of claim 130, wherein the subject is suffering from at least one of abdominal pain, pain in the mid-epigastric, chest, or back regions, gastrointestinal pain, difficulty breathing, loss of appetite, nausea, vomiting, inflammation of the pancreas, memory loss, dementia, xanthelasmas, corneal arcus, and xanthomas.

134. The method of any one of claims 130-133, wherein the subject has moderate hypertriglyceridemia associated with cardiovascular disease, atherosclerosis, obesity, hypertension, diabetes, insulin resistance, and/or liver disease; or severe hypertriglyceridemia associated with cardiovascular disease, atherosclerosis, obesity, hypertension, diabetes, insulin resistance, and/or liver disease.

135. A method of treating severe hypertriglyceridemia in a subject, comprising administering to the subject a therapeutically effective amount of the compound of formula (I) of claim 1, or a pharmaceutically acceptable salt thereof.

136. The method of claim 112, wherein the subject is suffering from at least one of abdominal pain, pain in the mid-epigastric, chest, or back regions, gastrointestinal pain, difficulty breathing, loss of appetite, nausea, vomiting, inflammation of the pancreas, memory loss, dementia, xanthelasmas, comeal arcus, and xanthomas.

137. The method of claim 135 or 136, wherein the subject has a triglyceride blood level above 500 mg/dL.

138. The method of any one of claims 135-137, wherein the subject has treatment resistant severe hypertriglyceridemia.

139. The method of any one of claims 135-138, wherein the subject has severe hypertriglyceridemia associated with cardiovascular disease, atherosclerosis, obesity, hypertension, diabetes, insulin resistance, and/or liver disease.

140. The method of any one of claims 135-139, wherein the subject is an adult male subject.

141. The method of any one of claims 135-140, wherein the subject is a Hispanic descendant.

142. The method of any one of claims 130-141, wherein the method comprises lowering low-density lipoprotein cholesterol levels and/or lowering non-high-density lipoprotein cholesterol levels.

143. The method of claim 142, wherein the method comprises at least one of: i) lowering triglyceride levels by at least 5%, at least 10%, or at least 20%; ii) lowering low-density lipoprotein cholesterol levels by at least 5%, at least 10%, or at least 20%; and iii) lowering non-high-density lipoprotein cholesterol levels by at least 5%, at least 10%, or at least 20%.

144. The method of any one of claims 130-143, wherein the method slows the progression of at least one of the cardiovascular disease, atherosclerosis, obesity, hypertension, diabetes, insulin resistance, and/or liver disease; and/or reduces the risk of a major cardiovascular event after the administration.

145. The method of claim 144, wherein the major cardiovascular event is death or hospitalization for worsening of the disease.

146. The method of any one of claims 130-145, wherein the method does not cause a clinically significant risk of adverse events in the subject after administration.

147. The method of claim 146 wherein the adverse events are associated with a mitochondria uncoupler.

148. The method of claim 147, wherein the mitochondria uncoupler is a phenolic metabolite of the compound of formula (I) according to claim 1.

149. The method of any one of claims 146-148, wherein the adverse events comprise at least one of nausea, vomiting, sweating, dizziness, headaches, cataracts, glaucoma, pyrexia, hyperthermia, tachycardia, diaphoresis, tachypnoea, and death.

150. The method of claim 149, wherein the adverse event is characterized by at least one of elevated body temperature, elevated heart rate, abnormal sweating, erythema, perspiration, dehydration, and abnormally rapid breathing.

151. The method of claim 149, wherein the adverse event is associated with cardiovascular collapse, cardiac arrest, and/or death.

152. The method of claim 149, wherein the adverse event is cardiac arrest.

153. A method of reducing liver fat by at least 50% in a subject, comprising administering to the subject a therapeutically effective amount of the compound of formula (I) of claim 1, or a pharmaceutically acceptable salt thereof.

154. The method of claim 153, wherein the subject is suffering from non-alcoholic fatty liver disease (NAFLD), non-alcoholic steatohepatitis (NASH), and/or hepatic steatosis.

155. The method of claim 153 or 154, further comprising the step of: determining Fibroscan® Vibration-controlled Transient Elastography (VCTE), Fibroscan® Controlled Attenuation Parameter (CAP) score, Magnetic resonance imaging proton density fat fraction (MRI-PDFF), and Enhanced Liver Fibrosis (ELF) score of the subject before and after administration.

156. The method of claim 155, wherein the subject has CAP score of greater than 300 dB/m before administration.

157. The method of claim 155, wherein the subject has at least 8% liver fat by MRI-PDFF before administering.

158. A method of reducing lipids by at least 10% in a subject, comprising administering to the subject a therapeutically effective amount the compound of formula (I) of claim 1, or a pharmaceutically acceptable salt thereof.

159. The method of claim 158, wherein the subject suffers from at least one of obesity, excess body fat, type 2 diabetes, insulin resistance or intolerance, high blood pressure, dyslipidemia, atherosclerosis, hypertriglyceridemia, acquired lipodystrophy, inherited lipodystrophy, partial lipodystrophy, metabolic syndrome, Rett's syndrome, metabolic syndrome associated with aging, metabolic diseases associated with increased reactive oxygen species (ROS), Friedreich's ataxia, NAFLD, NASH, noncirrhotic NASH, noncirrhotic NASH with liver fibrosis, hepatic steatosis, hepatic fibrosis, liver cirrhosis, and hepatocellular carcinoma.

160. The method of claim 158 or 159, further comprising the step of: determining serum high sensitivity C-reactive protein (hs-CRP), Lp(A), Apo B, low-density lipoprotein (LDL), high-density lipoprotein (HDL), total cholesterol, triglycerides, and free fatty acids (FFA) of the subject before and after administration.

161. The method of any one of claims 153-161, wherein the method provides at least one of: i) a reduction of body weight by at least 10%; ii) a reduction of HbAic by at least 0.5%, or at least 1.5%; iii) a reduction of blood pressure of at least 5 mmHg; iv) a reduction of lipids by at least 10%; v) a reduction of liver fat by at least 50%; vi) a reduction of serum alanine aminotransferase (ALT); and vii) a reduction of aspartate aminotransferase (AST).

162. The method of any one of claims 153-161, wherein the method does not cause a clinically significant risk of adverse events in the subject after administration.

163. The method of claim 162, wherein the adverse events are associated with a mitochondria uncoupler.

164. The method of claim 163, wherein the mitochondria uncoupler is a phenolic metabolite of the compound of formula (I) of claim 1.

165. The method of any one of claims 162-164, wherein the adverse events comprise at least one of nausea, vomiting, sweating, dizziness, headaches, cataracts, glaucoma, pyrexia, hyperthermia, tachycardia, diaphoresis, tachypnoea, and death.

166. The method of claim 165, wherein the adverse event is characterized by at least one of elevated body temperature, elevated heart rate, abnormal sweating, erythema, perspiration, dehydration, and abnormally rapid breathing.

167. The method of claim 165, wherein the adverse event is associated with cardiovascular collapse, cardiac arrest, and/or death.

168. The method of claim 167, wherein the adverse event associates cardiac arrest.

169. The method of any one of claims 135-168, wherein the method provides at least one of the following in the subject after administering about 30 mg to about 1400 mg of the phenolic metabolite of the compound of formula (I) of claim 1: i) a steady state of maximum plasma concentration (Cmax) of the phenolic metabolite from about 80 ng/ml to about 8300 ng/ml; ii) mean half-life (ini) of the phenolic metabolite of about 20-50 hours, about 25-40 hours, or about 30-40 hours; iii) median time to maximum plasma concentration (Tmax) of the phenolic metabolite of the compound of formula (I) about 6-8 hours or about 6-10 hours; iv) median area under the curve extrapolated to infinity (AUCinf) of the phenolic metabolite of about 3 h*μg/mL to about 420 h*μg/mL; and v) AUC/Cmax ratio of about 18.

170. A method of treating or reducing the risk of cancer in a subject, comprising administering to the subject a therapeutically effective amount of the compound of claim 1, or a pharmaceutically acceptable salt thereof.

171. The method of claim 170, wherein the cancer includes biliary tract cancer, bladder cancer, brain cancer (i.e., meningiomas), breast cancer (postmenopausal), cervical cancer, colorectal cancer, endometrial/uterine cancer, esophageal cancer, gallbladder cancer, head and neck cancer, kidney/renal cancer, leukemia, liver cancer, multiple myeloma, non-Hodgkin lymphoma, ovarian cancer, pancreatic cancer, stomach cancer and thyroid cancer, and prostate cancer.

172. The method of claim 170 or 171, wherein the cancer is associated with obesity, excess body fat, diabetes, high blood pressure, dyslipidemia, metabolic diseases, liver diseases, and/or cardiovascular diseases.

173. A method of treating obesity, excess body fat, type 2 diabetes, insulin resistance or intolerance, high blood pressure, dyslipidemia, atherosclerosis, hypertriglyceridemia, acquired lipodystrophy, inherited lipodystrophy, partial lipodystrophy, metabolic syndrome, Rett's syndrome, metabolic syndrome associated with aging, metabolic diseases associated with increased reactive oxygen species (ROS), Friedreich's ataxia, NAFLD, NASH, noncirrhotic NASH, noncirrhotic NASH with liver fibrosis, hepatic steatosis, hepatic fibrosis, liver cirrhosis, cancer, or hepatocellular carcinoma, the method comprising administering a therapeutically effective amount of the compound of formula (I) of claim 1 or a pharmaceutically acceptable salt thereof in a subject, to achieve at least one of: i) a steady state of maximum plasma concentration (Cmax) of a phenolic metabolite of the compound of formula (I) of from about 80 ng/mL to about 8300 ng/ml; ii) mean half-life (t½) of a phenolic metabolite about 20-50 hours, about 25-40 hours, or about 30-40 hours; iii) median time to maximum plasma concentration (Tmax) of the phenolic metabolite of about 6-8 hours or about 6-10 hours; iv) median area under the curve extrapolated to infinity (AUCmf) of the phenolic metabolite of about 3 h*μg/mL to about 420 h*μg/mL; and v) AUC/Cmax ratio of about 18.

174. The method of any one of proceeding claims, wherein the subject has elevated Body Mass Index (BMI).

175. The method of claim 175, wherein the subject has BMI of about 28.0 kg/m2 to about 45.0 kg/m2.

176. The method of any one of preceding claims, wherein the subject is in a fasted condition before administration.

177. The method of any one of preceding claims, wherein the subject is in a fed condition before administration.

178. The method of any one of preceding claims, wherein the therapeutically effective amount is from about 30 mg to about 1400 mg per day, from about 100 mg to about 1000 mg per day, from about 150 mg to about 600 mg per day, or from 200 mg to 550 mg per day.

179. The method of any one of preceding claims, wherein the therapeutically effective amount is about 30 mg, 50 mg, 75 mg, 100 mg, 150 mg, 170 mg, 200 mg, 250 mg, 300 mg, 340 mg, 350 mg, 400 mg, 450 mg, 500 mg, 510 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, or 1400 mg per day.

180. The method of claim 153, wherein the therapeutically effective amount is about 30 mg, 100 mg, 200 mg, 500 mg, 600 mg, 1050 mg, or 1400 mg per day.

181. The method of claim 153, wherein the therapeutically effective amount is about 200 mg, 400 mg, or 550 mg per day.

182. The method of claim 153, wherein the therapeutically effective amount is about 170 mg, 340 mg, 510 mg per day.

183. The method of claim 153, wherein the therapeutically effective amount is about 150 mg, 300 mg, 450 mg per day.

184. The method of any one of preceding claims, wherein the compound of formula (I) or a pharmaceutically acceptable salt thereof is administered orally once daily.

185. The method of any one of preceding claims, wherein the compound of formula (I) or a pharmaceutically acceptable salt thereof is administered once daily for two weeks, four weeks, six weeks, eight weeks, ten weeks, one month, two months, three months, four months, six months, eight month, or one year.

186. The method of any one of proceeding claims, wherein the compound of formula (I) of claim 1, or a pharmaceutically acceptable salt thereof, is administered in a hydroxypropyl methylcellulose capsule.
